(19) United States
(12) Patent Application Publication
(10) Pub. No.: US 2019/0033313 A1
(43) Pub. Date: Jan. 31, 2019

(54) METHODS AND SYSTEMS FOR DETECTING MELANOMA

(71) Applicant: University of Florida Research Foundation, Inc., Gainesville, FL (US)

(72) Inventors: NIKOLAUS GRAVENSTEIN, GAINESVILLE, FL (US); CHRISTIANA M. SHAW, GAINESVILLE, FL (US); MICHAEL T. COSTANZO, GAINESVILLE, FL (US); RICK YOST, GAINESVILLE, FL (US); CANDICE Z. ULMER, GAINESVILLE, FL (US); LOUIS SEARCY, GAINESVILLE, FL (US)

(21) Appl. No.: 15/579,078

(22) PCT Filed: Jun. 2, 2016
(86) PCT No.: PCT/US16/35442

§ 371 Date: Dec. 1, 2017

Related U.S. Application Data
(60) Provisional application No. 62/169,865, filed on Jun. 2, 2015.
(60) Provisional application No. 62/192,109, filed on Jul. 14, 2015.
(60) Provisional application No. 62/322,860, filed on Apr. 15, 2016.

(51) Int. Cl. G01N 33/574

(52) U.S. Cl. CPC G01N 33/5743; G01N 2405/00; G01N 2560/00

(57) ABSTRACT

The present disclosure provides methods and systems for detecting melanoma in a host. The methods include non-invasive methods of detecting melanoma and methods and systems for providing a molecular signature and/or melanoma biomarker signature for a host.

### CROSS-REFERENCE TO RELATED APPLICATIONS

This application is the 35 U.S.C. § 371 national stage application of PCT Application No. PCT/US2016/035442, filed Jun. 2, 2016, where the PCT claims the benefit of and priority to U.S. Provisional Patent Applications No. 62/169,865 filed on Jun. 2, 2015, entitled “METHODS AND SYSTEMS FOR DETECTING MELANOMA,” U.S. Provisional Patent Application No. 62/192,109 filed on Jul. 14, 2015 having the same title, and U.S. Provisional Patent Application No. 62/322,860 filed on Apr. 15, 2016 having the same title, the contents of each of which is incorporated by reference herein in its entirety.

### BACKGROUND

Early discovery of unique biological markers is a priority in all aspects of cancer-related research and clinical care. Cancer detection based on volatile analysis can be traced back to 1989, where a medical letter was published detailing a case where a dog noticed the presence of a malignant melanoma on its owner's skin. Current studies still focus on the use of animals and devices to “smell” cancer. Studies for detecting differences between the molecular and/or volatile signature from cancer cells vs. healthy cells require a series of time consuming and costly sample preparation steps.

Melanoma represents one of the most common and most serious skin cancers. Current approaches to melanoma screening and treatment rely on surgical removal of all suspicious skin lesions and/or a “watch-and-wait” approach. This causes many non-cancerous (normal) moles to be unnecessarily removed and also misses many malignant moles. Reliance on the inexact art of melanoma identification by mole appearance hinders early detection and also results in many unnecessary biopsies.

### SUMMARY

The present disclosure provides methods and systems for detecting melanoma in a host. In embodiments, methods of detecting melanoma in a host include non-invasively obtaining a sample from a skin lesion of a host where the lesion is suspected of including melanoma, analyzing the sample with a chemical analysis technique, and determining the presence and/or amount in the sample of a set of identified melanoma biomarkers selected based on multivariate data analysis of melanoma and non-melanoma samples. The methods further include providing a biomarker signature of the host sample based on the chemical analysis of the sample, where the biomarker signature indicates the presence and/or amount of one or more of the melanoma biomarkers, comparing the biomarker signature of the host sample to a biomarker signature of a control sample, and making a preliminary patient diagnosis of melanoma based on the comparison of the host sample biomarker signature and the control sample biomarker signature. In embodiments, the chemical analysis technique is selected from: mass spectrometry, liquid chromatography, gas chromatography, ion mobility spectrometry, and high-field asymmetric waveform ion mobility spectrometry.

In embodiments, methods of the disclosure described above also include determining whether at least one compound selected from the group of compounds from group (A) consisting of: lactic acid, a compound correlated to the m/z value 86.096, 90.055, benzaldehyde, a compound correlated to the m/z value 109.065, proline, betaine, the compound correlated to the m/z value 120.081, leucine or isoleucine, octanoic acid, L-methionine S-oxide, spermidine, methionine, phenylalanine, phosphocholine, a compound correlated to the m/z value 188.071, acetyl spermidine, the compound correlated to the m/z value 192.069, thiopurine, a compound correlated to the m/z value 204.123, tryptophan, carmustine, n-formylmethionine, a compound correlated to the m/z value 225.097, 4-(glutamylamino) butanoate, propionyl-L-carnitine, phosphatidylglycerol, aspartyl-gamma-glutamate, aspartyl-glutamine, DOPA sulfate, alanine-leucine-proline, diacetyl spermine, inosine 5′-monophosphate, a lipid correlated to the m/z value 568.363, a lipid correlated to the m/z value 700.530, a lipid correlated to the m/z value 750.535, and a lipid correlated to the m/z value 854.595 is overexpressed in the host volatile sample; and/or determining whether at least one compound selected from the group of compounds from group (B) consisting of: succinic anhydride, benzoic acid, propylene glycol, histidine, octadienoic acid, 4-hydroxy-L-glutamic acid, methylhistidine, glycine-proline, aminomuconic acid, N-acetyl-L-glutamic acid, 5-hydroxyindoleacetic acid, 191.089, tetrahydrodipicolinate, 208.116, glutamylalanine, 5-(4-chloro-3-hydroxy-1-butynyl)-2,2′-bithiophene, ethyl glucuronide, 288.253, 310.235, skullcapflavone I, trihydroxyoctadecenoic acid, adenosine monophosphate, alanine-glycine-histidine-proline, a lipid correlated to the m/z value 560.369, a lipid correlated to the m/z value 810.599, a lipid correlated to the m/z value 818.727, and a lipid correlated to the m/z value 879.735 is underexpressed in the host volatile sample; and diagnosing the host as having melanoma or a risk of developing melanoma if at least one of the compounds from group (A) is overexpressed and/or at least one of the compounds from group (B) is underexpressed, when compared to a mean expression level of the compound(s) in one or more control samples.

Systems of the present disclosure for real-time detection of melanoma in a host, in embodiments, include: a device for provoking an emission from a skin lesion of a host to produce an emission sample from the host, where the lesion is suspected of including melanoma; a sample reservoir for containing or capturing the emission sample, wherein the sample reservoir is configured to interface with a mass spectrometry device; a mass spectrometry device capable of analyzing the chemical content of the emission sample; and a signal processing mechanism in operative communication with the mass spectrometry device, the signal processing mechanism having data transfer and evaluation software protocols configured to transform raw data from the mass spectrometry device into information regarding melanoma biomarkers, wherein the information comprises one or more of the presence, absence, and amounts of the melanoma biomarkers in the emission sample. In embodiments, the signal processing mechanism produces a biomarker signature corresponding to the host sample.

In the present disclosure, some embodiments of methods of diagnosing or monitoring melanoma in a host include the following steps: obtaining a sample from a skin lesion of the host, wherein the lesion is suspected of including melanoma; analyzing the sample with a chemical analysis technique selected from the group consisting of: mass spectrometry, liquid chromatography, gas chromatography, ion mobility spectrometry, and high-field asymmetric waveform ion mobility spectrometry to determine a molecular signature of the sample; detecting the presence and/or amount of one or more molecular melanoma biomarkers from the molecular signature of the sample, wherein the molecular melanoma biomarkers are selected from the group of compounds consisting of: lactic acid, a compound correlated to the m/z value 86.096, a compound correlated to the m/z value 90.055, benzaldehyde, a compound correlated to the m/z value 109.065, proline, succinic anhydride, betaine, a compound correlated to the m/z value 120.081, benzoic acid, leucine or isoleucine, propylene glycol, histidine, octanoic acid, L-methionine S-oxide, spermidine, methionine, octadienoic acid, 4-hydroxy-L-glutamic acid, phenylalanine, methylhistidine, glycine-proline, aminomuconic acid, phosphocholine, N-acetyl-L-glutamic acid, a compound correlated to the m/z value 188.071, acetyl spermidine, 5-hydroxyindoleacetic acid, a compound correlated to the m/z value 191.089, a compound correlated to the m/z value 192.069, tetrahydrodipicolinate, thiopurine, a compound correlated to the m/z value 204.123, tryptophan, a compound correlated to the m/z value 208.116, carmustine, n-formylmethionine, a compound correlated to the m/z value 225.097, 4-(glutamylamino) butanoate, glutamylalanine, propionyl-L-carnitine, phosphatidylglycerol, 5-(4-Chloro-3-hydroxy-1-butynyl)-2,2′-bithiophene, ethyl glucuronide, aspartyl-gamma-glutamate, aspartyl-glutamine, DOPA sulfate, a compound correlated to the m/z value 288.253, alanine-leucine-proline, diacetyl spermine, a compound correlated to the m/z value 310.235, skullcapflavone I, trihydroxyoctadecenoic acid, adenosine monophosphate, inosine 5′-monophosphate, alanine-glycine-histidine-proline, and the group of lipids corresponding to the group of m/z values consisting of: 560.369, 568.363, 700.530, 750.535, 810.599, 818.727, 854.595, and 879.735; comparing the amount of the one or more molecular melanoma biomarkers from the host sample an amount of the same one or more molecular melanoma biomarkers from a molecular signature of a control sample, and making a preliminary diagnosis of melanoma when one or more molecular melanoma biomarkers are present in a concentration significantly greater than or significantly less than the concentration in the control sample.

Additional methods of the present disclosure for detecting melanoma in a host include: obtaining a sample from a skin lesion of a host, wherein the lesion is suspected of including melanoma; analyzing the sample with a mass spectrometry or ion mobility technique to determine a lipid signature of the sample; detecting the presence and/or amount of one or more lipid melanoma biomarkers from the lipid signature of the sample, wherein the lipid melanoma biomarkers are selected from the group of lipids corresponding to the group of m/z values consisting of: 560.369, 568.363, 700.530, 750.535, 810.599, 818.727, 854.595, 879.735, 484.436, 594.485, 798.634, 804.590, 817.638, 819.519, 820.585, 822.538, 845.534, 878.591, 890.665, 898.726, 479.393, 677.549, 781.551, 821.667, 832.742, 879.736, and 917.831; comparing the amount of the one or more lipid melanoma biomarkers in the host sample to the amount of the same one or more lipid melanoma biomarkers from a lipid signature of a control sample; and determining a potential diagnosis of melanoma when one or more lipid melanoma biomarkers are present in a concentration significantly greater than or significantly less than the concentration in the control sample.

The details of some embodiments of the present disclosure are set forth in the description below. Other features, objects, and advantages of the present disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Figure descriptions appear in the Examples, below.

FIGS. 1A-1C illustrate principal component analysis (PCA) and partial least squares discriminant analysis (PLSDA) score plots and PLSDA loadings plots for molecular samples from Example 1. FIG. 1A shows the principal component analysis (PCA) of the samples, and FIG. 1B shows the partial least squares discriminant analysis (PLSDA) score plots. FIG. 1C is a PLSDA loadings plot showing first component separation of normal (upper numbers) cells vs. melanoma (lower).

FIG. 2 illustrates total ion chromatogram (TIC) for LC/HESI/HRMS of normal cells. The inset is a TIC from 1-5.5, 9.5-11 min for normal (lower plot) cells and pooled male/female melanoma (upper plot) cells.

FIG. 3 illustrates PLSDA score and loadings plots showing first component separation of normal cells (upper points/oval) against pooled male/female melanoma (lower points/oval) cells.

FIGS. 4A-B illustrate PLSDA score and loadings plots showing first component separation of normal cells vs. male and female melanoma cells (FIG. 4A), and PLSDA score plot showing first component separation of male vs. female melanoma cells (FIG. 4B).

FIG. 5 illustrates a PC analysis scores plot displaying first component separation of normal cells (triangles, top oval) vs. melanoma cells (lower ovals). Of the lower, melanoma cells, female (F), male (M), and hybrid male/female (MF) are shown.

FIGS. 6A and 6B illustrate m/z values of various biomarkers. FIG. 6 A is a plot of mean decreased accuracy (MDA) of the 15 m/z features from UHPLC/HRMS analysis that contribute most to the random forest training set. FIG. 6B is a plot of the top 25 m/z features from UHPLC/HRMS analysis with the greatest variable information processing (VIP) scores from PLS-DA. The column on the right of each plot indicates whether a given feature is up or down regulated in melanoma vs normal samples.

FIG. 7 illustrates PLSDA score and loadings plot showing first component separation of normal cells against pooled male/female melanoma cells.

FIGS. 8A-8B illustrate PLSDA scores plot (8A) and loadings plot (8B) showing first component separation of normal vs. male and female melanoma biopsied human skin tissue.

FIG. 9 illustrates a PCA scores plot for samples from Example 3 showing separation between melanoma and the control tissue samples within the first principal component.

FIG. 10 is a hierarchical clustering plot using PLS-DA for compound identification.

FIG. 11 is a SAM plot illustrating that 45 metabolite features fall within the positive and negative standard deviation distribution estimated by random permutations of the dataset.

FIG. 12 is a volcano plot of the metabolite data from Example 3.

FIG. 13 is a PCA scores plot illustrating separation between melanoma and the control tissue samples from Example 4 within the first principal component.

FIG. 14 is a hierarchical clustering plot using PLS-DA for compound identification.

FIG. 15 is a SAM plot illustrating that 65 metabolite features fall within the positive and negative standard deviation distribution estimated by random permutations of the dataset.

FIG. 16 is a PCA scores plot of the tissue samples from Example 4 showing principal component separation between the lipid portion of melanoma and normal skin tissue.

FIG. 17 is a PLS-DA scores plot (FIG. 17) illustrating separation between melanoma and the control tissue samples within the first principal component.

FIG. 18 is a variable importance in projection (VIP) score plot showing the top 30 features.

FIG. 19 is a random forest mean decrease accuracy (MDA) plot showing the top 15 features.

FIG. 20 is a SAM plot of flowprobe data showing that 489 lipid features fall within the positive and negative standard deviation distribution estimated by random permutations of the dataset are provided here.

FIG. 21 is a graph illustrating an intensity trend for ceremides (Cer) in melanoma tissue vs. normal tissue.

FIG. 22 is a graph illustrating an intensity trend for lisophophatidylcholines (LPC) in melanoma tissue vs. normal tissue.

FIG. 23 is a graph illustrating an intensity trend for phophatidylcholines (PC) in melanoma tissue vs. normal tissue.

FIG. 24 is a graph illustrating an intensity trend for phosphatidylethanolamines (PE) in melanoma tissue vs. normal tissue.

FIG. 25 is a graph illustrating an intensity trend for sphingomyelins (SM) in melanoma tissue vs. normal tissue.

FIGS. 26A-B are graphs illustrating an intensity trend for triacylglycerides (TAG) in melanoma vs normal tissues.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

Of the publications and patents cited in this specification, those that are incorporated by reference are specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of analytical chemistry, physical chemistry, molecular biology, organic chemistry, biochemistry, genetics, statistical analysis, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “ includes,” “including,” and the like; “consisting essentially of” or “consists essentially” or the like, when applied to methods and compositions encompassed by the present disclosure refers to compositions like those disclosed herein, but which may contain additional structural groups, composition components or method steps. Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Definitions:

In describing and claiming the disclosed subject matter, the following terminology will be used in accordance with the definitions set forth below.

The term “organism,” “subject,” or “host” refers to any living entity, including humans, mammals (e.g., cats, dogs, horses, mice, rats, pigs, hogs, cows, and other cattle), birds (e.g., chickens), and other living species that are in need of treatment. In particular, the term “host” includes humans. As used herein, the term “human host” or “human subject” is generally used to refer to human hosts.

The term “cancer”, as used herein, shall be given its ordinary meaning, as a general term for diseases in which abnormal cells divide without control and form cancer or neoplastic cells or tissues. The term cancer can include cancer cells, neoplasms and/or precancerous cells. Cancer cells can invade nearby tissues and can spread through the bloodstream and lymphatic system to other parts of the body. There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system. Melanoma is a cancer typically formed from pigment containing cells in the skin.

When normal cells lose their ability to behave as a specified, controlled and coordinated unit, a tumor may be formed. Generally, a solid tumor is an abnormal mass of tissue that usually does not contain cysts or liquid areas but may include necrotic areas. Different types of solid tumors are named for the type of cells that form them. Examples of solid tumors are sarcomas, carcinomas, melanomas, and lymphomas. Leukemias (cancers of the blood) generally do not form solid tumors.

Representative cancers include, but are not limited to, melanoma, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain tumors: astrocytoma, glioblastoma, medulloblastoma, and ependymoma, Ewing's sarcoma family of tumors, germ cell tumor, extracranial cancer, Hodgkin's disease, leukemia, acute lymphoblastic leukemia, acute myeloid leukemia, liver cancer, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult.

Benign tumors have less of a tendency to invade and are less likely to metastasize. Depending on their location, they can be just as life threatening as malignant lesions. An example of this would be a benign tumor in the brain such as a meningioma, which can grow and occupy space within the skull, leading to increased pressure on the brain.

It should be noted that precancerous cells, cancer, tumors are often used interchangeably in the disclosure.

As used herein, “isolated” means removed or separated from the native environment. An isolated compound, peptide or protein (e.g., an enzyme) indicates the protein is separated from its natural environment. Isolated peptides or proteins are not necessarily purified. An “isolated sample” is a sample (e.g., in liquid, solid, or vapor form) obtained from and removed or separated from that host.

As used herein, the term “removed” or “substantially removed” indicates that an amount of a substance or compound has been separated from another composition, but does not require that absolutely all traces of the removed substance be absent from the remaining composition, such that the removed substance is completely undetectable.

The term “polypeptides” and “protein” include proteins, such as enzymes, and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

Modifications and changes can be made in the structure of the polypeptides in the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

“Identity,” as known in the art, is a relationship between two or more polypeptide sequences, as determined by comparing the sequences. In the art, “identity” also refers to the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

The terms “native,” “wild type”, or “unmodified” in reference to an organism (e.g., plant or cell), polypeptide, protein or enzyme, are used herein to provide a reference point for a variant/mutant of an organism, polypeptide, protein, or enzyme prior to its mutation and/or modification (whether the mutation and/or modification occurred naturally or by human design). Typically, the unmodified, native, or wild type organism, polypeptide, protein, or enzyme has an amino acid sequence that corresponds substantially or completely to the amino acid sequence of the polypeptide, protein, or enzyme as it generally occurs naturally.

As used herein, the term “control sample” refers to a sample obtained from a host or site on a host that does not have the condition being examined (e.g., melanoma). In embodiments, “control sample” may refer to a hypothetical “sample” with values determined from a mean of values of multiple samples from hosts or host sites without melanoma.

The term “volatile compound”, “volatile”, or “volatile organic compound (VOC)” refers to certain chemical volatile and semi-volatile compounds that have a high vapor pressure and/or low boiling point at room temperature and can thus easily evaporate or sublimate from the liquid or solid to the vapor form, which can be detected in the surrounding air by certain devices, some of which can be smelled by humans or other animals (e.g., dogs). As used herein, volatile also includes “semi-volatile” compounds that may need a slight provocation for release from solid or liquid form.

As used herein, the term “biomarker” refers to a compound (e.g., volatile, semi-volatile, or non-volatile compound) obtained from a subject that indicates (by virtue of its presence/absence or threshold quantity in the sample) a certain biological condition or lack thereof, such as a disease (e.g., cancer, such as melanoma). Thus, a “melanoma biomarker” refers to a compound that, by virtue of its presence, absence, or quantity in a sample from a subject indicates that the subject has or does not have melanoma.

As used herein, the term “biomarker signature” refers to a specific signature of a set (e.g., two or more, three or more, four or more, and so on) of identified biomarkers that is specific to an individual subject/host or a specified sample from an individual subject. The biomarker signature for a specific sample from a subject may include the presence/absence/quantity/ratio, etc. regarding a set of identified biomarker compounds. Similarly, the term “molecular signature” refers to a specific signature of a set (e.g., two or more, three or more, four or more, and so on) of molecules specific to an individual subject and/or sample from an individual subject. A “molecular signature” may or may not include biomarkers and can include additional compounds that may not be biomarkers. A “biomarker signature” may include additional compounds that are not biomarkers, but does include an indication of an amount (from the subject sample) of at least one identified biomarker.

As used herein, the term “m/z value” refers to the mass-to-charge ratio of an ion produced in an ionization source, as measured by a mass spectrometer. This term can also be extended to other analytical methods, such as, but not limited to ion mobility, that analyze ions produced in an ionization source and can refer to the collision cross section, drift time, or compensation voltage (CV) of an ion.

The term “sample” can refer to a tissue sample, cell sample, a fluid sample, vapor sample, and the like. A sample may be taken from a host. The tissue sample can include hair (including roots), buccal swabs, blood, saliva, semen, skin, muscle, or from any internal organs. The fluid may be, but is not limited to, urine, blood, ascites, pleural fluid, spinal fluid, semen, wound exudates, skin lesion, sputum, fecal matter, saliva, and the like. A vapor sample can include, breath, other gaseous exudates, vapor samples provoked from skin or other tissues, and the like. The body tissue can include, but is not limited to, skin, muscle, endometrial, uterine, and cervical tissue. A sample, in the context of the present disclosure, is primarily a biological sample (e.g., from a living host). Although a liquid sample and some solid samples may be used as a test sample without modification for testing directly, if a solid sample is to be made into liquid or vapor form for testing and/or a liquid sample is to be diluted or made into vapor form, a test sample may be made by reconstituting, dissolving, diluting, evaporating or sublimating the sample, and the like. In embodiments of the present disclosure “samples” refers to a substance gathered/obtained from a host/subject. In embodiments the sample may be gaseous, solid, or liquid samples and may include volatile, semi-volatile, and/or non-volatile compounds. In some instances, such a sample is referred to as a “molecular sample” or “host sample” and in some embodiments, where the sample is primarily gaseous and contains volatile compounds, the sample may be referred to as “volatile sample”. In embodiments, the samples are obtained from a host by non-invasive means (e.g., without surgical removal of tissue from the subject).

The term “detectable” refers to the ability to perceive or distinguish a signal (e.g., a specific compound or combination of compounds (e.g., a signature)) over a background signal. “Detecting” refers to the act of determining the presence of a target or the occurrence of an event by perceiving a signal that indicates the presence of a target or occurrence of an event, where the signal is capable of being perceived over a background signal.

As used herein, the term “provoking an emission” of compounds (e.g., volatile, semi-volatile, or non-volatile compounds) from a sample indicates that a molecular signal is induced or enhanced by performing a type of action on the surface of the skin of the host (e.g., a mole or skin lesion suspected of potential to be a melanoma), where the action increases the release of compounds from the skin surface of the host as compared to the amount of compounds released without the action. In embodiments, provoking the signal is analogous to a “scratch and sniff” sticker (e.g., scratching or rubbing the sticker produces a more pronounced and detectable odor due to release of compounds than in the absence of the scratching). In embodiments, provoking the signal can also include actions such as, but not limited to, local heating, inducing by laser, applying a vacuum, use of a solvent as a liquid or swab, DESI (which uses a spray of charged droplets to provoke emission), DART (which uses a beam of energetic atoms or molecules to provoke emission), flow probe, heat or light form a small light source (e.g., LED or laser LED), and the like.

An “emission” refers to a giving-off/release of compounds from the skin of the host. The emission may be gaseous (e.g., volatile compounds), but may also be in other forms, such as small solid particles or liquid and may include non-volatile compounds such as lipids and other molecular biomarkers.

Having defined some of the terms herein, the various embodiments of the disclosure will be described.

### Description:

Embodiments of the present disclosure include methods and systems for detecting forms of cancer, such as melanoma and other cancers, by analysis (e.g., real-time analysis) of various compounds (e.g., volatile, semi-volatile, and non-volatile compounds) present in a sample, or the air surrounding a sample, where the sample was obtained from a patient/host suspected of having or being screened for cancer.

Utilization of ambient ionization and separation methods may allow real-time, direct analysis of biomarkers for melanoma and other cancers, in addition to identification of new markers. Recent studies observed an apparent difference between the volatile metabolome of melanoma (cancer) and melanocyte (normal) cells, but only after a series of time consuming sample preparation steps. Consequently, analyzing metabolites in a more native environment (i.e., diseased tissue samples and swabs of potentially cancerous skin lesions) could lead to a greater number of, and more accurate, biomarkers. Therefore, the identification of additional biomarkers, as well as the development of a real-time method incorporating little or no sample preparation, would revolutionize current melanoma and other disease detection schemes.

Thus, the present disclosure provides methods and systems for screening for/detecting the presence of skin cancer (e.g., melanoma) in a host by detecting and analyzing the molecular signature (e.g., of volatile, semi-volatile, and/or non-volatile compounds) from a sample from the host. In embodiments, the host sample is analyzed in real time. In embodiments, no advanced treatment of the host sample is performed prior to analysis. For instance, in embodiments, direct sampling methods such as, but not limited to, DESI and DART are used to directly sample/analyze the host sample without any pretreatment of the host skin or tissue.

In embodiments, methods of detecting melanoma in a host include the steps of obtaining a sample from a skin lesion of a host (such as a suspected melanoma or a lesion suspected of having pre-cancerous cells) and analyzing the sample from the host for the presence of one or more biomarkers for melanoma. The method can further include detecting the amount of one or more of the biomarkers from the host sample and comparing the amount of melanoma biomarkers in the host sample to an amount of the melanoma biomarkers in a control sample or in a “standard” or “control” signal produced from a mean of amounts of certain melanoma biomarkers in multiple control (e.g., melanoma free) samples. In embodiments, the method includes determining a diagnosis, potential diagnosis, or preliminary diagnosis of melanoma or other cancer when there is a statistically significant difference between the amount of one or more melanoma or other cancer biomarkers in the host sample and the amount of the same melanoma or other cancer biomarker(s) in a control or “standard” sample or signature. A preliminary diagnosis of melanoma or cancer indicates that the result of the analysis of the molecular/biomarker signature of the sample is positive for melanoma or inconclusive, but that additional testing may be indicated before a confirmed diagnosis of melanoma is made.

In embodiments, the sample is a vapor (volatile), semi-volatile or non-volatile sample or some combination thereof obtained by provoking an emission (e.g., a molecular emission, a gaseous emission of compounds, etc.) from a skin surface of the host. In such embodiments, an emission of a molecular signature (metabolites, lipids, volatile, non-volatile, etc.) is induced or enhanced by performing an action on the surface of the skin of the host (e.g., a mole or skin lesion suspected of potential to be a melanoma), where the action increases the release of compounds from the skin surface of the host thereby providing an emission, or emission sample. In embodiments, the sample is obtained directly from the host (e.g., from the skin of the host), but in other embodiments, the sample may be taken from a sample previously obtained from the host (e.g., a removed skin or other tissue sample, such as a mole biopsy or a tissue swab containing material from the host).

In embodiments the action used to provoke the emission can include friction, such as physical contact of the skin with a physical object (e.g., swabbing, rubbing, abrasion, etc. with a sample collection material, such as, but not limited to a swab, cloth, etc.). In embodiments, the signal is provoked by friction caused by bombarding the host or sample with small particles, such as atoms or ions (e.g., such as but not limited to, spray ionization techniques such as desorption electrospray ionization (DESI)). In other embodiments, the signal is provoked by suction/aspiration of the skin or sample (e.g., a small vacuum suction device place over the skin of the host or sample. Other non-invasive methods and devices known in the art can be used to provoke a molecular emission from a subject.

In embodiments, the molecular sample is collected (directly from the host or from a previously obtained sample or swab from the host), and contained in a container or reservoir adapted for housing the sample. The host sample contains various compounds (e.g., volatile, semi-volatile, and/or non-volatile) from the host and can be analyzed with a chemical analysis technique to identify the presence and/or amount in the sample of a set of identified melanoma biomarkers that indicate the presence (cancer) or absence (normal) of cancerous cells (e.g., melanoma). In embodiments the sample includes molecular metabolites and/or lipid compounds that are melanoma biomarkers. This analysis is used to produce a molecular signature and/or biomarker signature for the host sample, where the signature indicates the presence and/or amount of one or more of the melanoma biomarkers. In some embodiments the sample is a gaseous/vapor sample and is contained in a container/reservoir adapted for housing a vapor sample. Such vapor samples contain volatile compounds from the host source that can then be analyzed for the presence of particular volatile compounds or a combination of volatile compounds in certain amounts and proportions (e.g., a volatile signature) that indicate the presence (cancer) or absence (normal) of cancerous cells (e.g., melanoma).

In embodiments, the host sample is analyzed directly by mass spectrometry or other chemical analysis technique. In embodiments the chemical analysis technique includes, but is not necessarily limited to, mass spectrometry, liquid chromatography, gas chromatography, ion mobility spectrometry, and high-field asymmetric waveform ion mobility spectrometry. It is desirable for the sample to be analyzed in real-time (e.g., in a clinical setting, in-office), thus, in embodiments, the sample is analyzed by a portable mass spec, such as an ion-mobility separation (IMS) mass spec or high-field asymmetric ion mobility spectrometry (FAIMS). This allows clinical diagnosis in real time without the need for additional sample pre-preparation. In embodiments, the analysis can also be validated in real time or later by additional detection techniques such as liquid chromatography or high-resolution mass spectrometry. This allows real-time analysis and diagnosis of skin cancer in an office setting with the option of later validation using additional techniques that may need to be performed in a lab setting.

In embodiments, methods of detecting melanoma in a patient include obtaining a sample from a host, as described above, and analyzing the sample to detect the presence and/or amount of compounds in the sample that are associated with melanoma (melanoma biomarkers). In embodiments the sample obtained from the host is analyzed with mass spectrometry to determine a molecular signature of the sample. The molecular signature is a representation (e.g., chart, graph, etc.) of the concentrations/amounts of various compounds present in the sample. In embodiments, the molecular signature is produced by a mass spectrometry device or by a signal processing mechanism in operative communication with the mass spectrometry device, where the signal processing mechanism has data transfer and evaluation software protocols configured to transform raw MS data from the mass spectrometry device into information correlated to (e.g., the m/z value in the MS data can be correlated to the identified compounds by ionization pattern(s) of the identified compound) the identified compounds and/or certain identified melanoma biomarkers or other cancer biomarkers.

In embodiments the presence/absence, amounts/concentrations of various compounds (e.g., biomarkers and other compounds) can be determined from the molecular signature of the sample (e.g., the m/z signature that correlates to the identified compound). In embodiments the molecular signature (or information obtained from the molecular signature, for example m/z pattern) is compared to the molecular signature of a control sample (e.g., from a host not having melanoma or from a skin area not having melanoma) or to an average control molecular signature prepared from taking mean values of biomarkers and other compounds obtained from molecular signatures of multiple control samples. In embodiments, the amounts of one or more specific compounds may be directly determined and compared without preparation of a molecular signature.

In embodiments, certain compounds are present, or present in statistically significant greater or lesser concentrations in melanoma or other cancer samples as compared to control samples, and these certain compounds are identified as melanoma biomarkers or other cancer biomarkers. Some cancer biomarkers are present in higher concentrations (e.g., upregulated/overexpressed) in cancer samples than in normal cells, whereas other cancer biomarkers are present in lower concentrations (e.g., down-regulated/underexpressed) in cancer samples than in normal cells. In embodiments melanoma biomarkers are identified by their m/z value (or m/z pattern) as determined by mass spectrometry or other chemical analysis technique. In embodiments, the presence of one or more melanoma biomarkers in a statistically significant different amount in a host sample (as compared to a control sample) indicates the probable presence of melanoma cells in the host. In embodiments, melanoma biomarkers of the present disclosure include, but are not limited to, those compounds listed in the Examples below (e.g., Examples 1-4, tables 1, 2, 4 and 5).

In embodiments, identified melanoma biomarkers include the following compounds (volatile compounds, lipid compounds, etc.) (where compound names are un-identified, biomarkers are correlated to m/z values), as provided in the corresponding examples below and Tables 1, 2, 4, and 5: lactic acid, a compound correlated to the m/z value 86.096, a compound correlated to the m/z value 90.055, benzaldehyde, a compound correlated to the m/z value 109.065, proline, succinic anhydride, betaine, a compound correlated to the m/z value 120.081, benzoic acid, leucine or isoleucine, propylene glycol, histidine, octanoic acid, L-methionine S-oxide, spermidine, methionine, octadienoic acid, 4-hydroxy-L-glutamic acid, phenylalanine, methylhistidine, glycine-proline, aminomuconic acid, phosphocholine, N-acetyl-L-glutamic acid, a compound correlated to the m/z value 188.071, acetyl spermidine, 5-hydroxyindoleacetic acid, a compound correlated to the m/z value 191.089, a compound correlated to the m/z value 192.069, tetrahydrodipicolinate, thiopurine, a compound correlated to the m/z value 204.123, tryptophan, a compound correlated to the m/z value 208.116, carmustine, n-formylmethionine, a compound correlated to the m/z value 225.097, 4-(glutamylamino) butanoate, glutamylalanine, propionyl-L-carnitine, phosphatidylglycerol, 5-(4-Chloro-3-hydroxy-1-butynyl)-2,2′-bithiophene, ethyl glucuronide, aspartyl-gamma-glutamate, aspartyl-glutamine, DOPA sulfate, a compound correlated to the m/z value 288.253, alanine-leucine-proline, diacetyl spermine, a compound correlated to the m/z value 310.235, skullcapflavone I, trihydroxyoctadecenoic acid, adenosine monophosphate, inosine 5′-monophosphate, alanine-glycine-histidine-proline, and the group of lipids corresponding to the group of m/z values consisting of: 560.369, 568.363, 700.530, 750.535, 810.599, 818.727, 854.595, and 879.735.

In embodiments, melanoma biomarkers that are present in greater amounts (or overexpressed) in melanoma samples include, but are not limited to the following compounds: lactic acid, a compound correlated to the m/z value 86.096, 90.055, benzaldehyde, a compound correlated to the m/z value 109.065, proline, betaine, the compound correlated to the m/z value 120.081, leucine or isoleucine, octanoic acid, L-methionine S-oxide, spermidine, methionine, phenylalanine, phosphocholine, a compound correlated to the m/z value 188.071, acetyl spermidine, the compound correlated to the m/z value 192.069, thiopurine, a compound correlated to the m/z value 204.123, tryptophan, carmustine, n-formylmethionine, a compound correlated to the m/z value 225.097, 4-(glutamylamino) butanoate, propionyl-L-carnitine, phosphatidylglycerol, aspartyl-gamma-glutamate, aspartyl-glutamine, DOPA sulfate, alanine-leucine-proline, diacetyl spermine, inosine 5′-monophosphate, a lipid correlated to the m/z value 568.363, a lipid correlated to the m/z value 700.530, a lipid correlated to the m/z value 750.535, and a lipid correlated to the m/z value 854.595.

In embodiments, biomarkers that are downregulated/underexpressed in melanoma (e.g., present in higher amounts in normal cells) include, but are not limited to, the following compounds: lactic acid, a compound correlated to the m/z value 86.096, 90.055, benzaldehyde, a compound correlated to the m/z value 109.065, proline, betaine, the compound correlated to the m/z value 120.081, leucine or isoleucine, octanoic acid, L-methionine S-oxide, spermidine, methionine, phenylalanine, phosphocholine, a compound correlated to the m/z value 188.071, acetyl spermidine, the compound correlated to the m/z value 192.069, thiopurine, a compound correlated to the m/z value 204.123, tryptophan, carmustine, n-formylmethionine, a compound correlated to the m/z value 225.097, 4-(glutamylamino) butanoate, propionyl-L-carnitine, phosphatidylglycerol, aspartyl-gamma-glutamate, aspartyl-glutamine, DOPA sulfate, alanine-leucine-proline, diacetyl spermine, inosine 5′-monophosphate, a lipid correlated to the m/z value 568.363, a lipid correlated to the m/z value 700.530, a lipid correlated to the m/z value 750.535, and a lipid correlated to the m/z value 854.595.

In embodiments, a method of detecting melanoma in a host includes obtaining the sample and detecting melanoma biomarkers as described above and determining whether at least one compound selected from the group of compounds from group (A) is overexpressed in the host volatile sample, where group (A) includes the following compounds: lactic acid, a compound correlated to the m/z value 86.096, 90.055, benzaldehyde, a compound correlated to the m/z value 109.065, proline, betaine, the compound correlated to the m/z value 120.081, leucine or isoleucine, octanoic acid, L-methionine S-oxide, spermidine, methionine, phenylalanine, phosphocholine, a compound correlated to the m/z value 188.071, acetyl spermidine, the compound correlated to the m/z value 192.069, thiopurine, a compound correlated to the m/z value 204.123, tryptophan, carmustine, n-formylmethionine, a compound correlated to the m/z value 225.097, 4-(glutamylamino) butanoate, propionyl-L-carnitine, phosphatidylglycerol, aspartyl-gamma-glutamate, aspartyl-glutamine, DOPA sulfate, alanine-leucine-proline, diacetyl spermine, inosine 5′-monophosphate, a lipid correlated to the m/z value 568.363, a lipid correlated to the m/z value 700.530, a lipid correlated to the m/z value 750.535, and a lipid correlated to the m/z value 854.595. In some embodiments, the method includes determining whether at least one compound selected from the group of compounds from group (B) is underexpressed in the host volatile sample, where group (B) includes the following compounds: succinic anhydride, benzoic acid, propylene glycol, histidine, octadienoic acid, 4-hydroxy-L-glutamic acid, methylhistidine, glycine-proline, aminomuconic acid, N-acetyl-L-glutamic acid, 5-hydroxyindoleacetic acid, a compound correlated to the m/z value 191.089, tetrahydrodipicolinate, a compound correlated to the m/z value 208.116, glutamylalanine, 5-(4-chloro-3-hydroxy-1-butynyl)-2,2′-bithiophene, ethyl glucuronide, a compound correlated to the m/z value 288.253, a compound correlated to the m/z value 310.235, skullcapflavone I, trihydroxyoctadecenoic acid, adenosine monophosphate, alanine-glycine-histidine-proline, a lipid correlated to the m/z value 560.369, a lipid correlated to the m/z value 810.599, a lipid correlated to the m/z value 818.727, and a lipid correlated to the m/z value 879.735.

In embodiments, the method includes determining whether any group (A) compounds are overexpressed in the host sample and determining whether any group (B) compounds are underexpressed in the host sample. In embodiments, the method further includes diagnosing the host as having melanoma or a risk of developing melanoma if at least one of the compounds from group (A) is overexpressed and/or at least one of the compounds from group (B) is underexpressed, when compared to a mean expression level of the compound(s) in one or more control samples.

In embodiments, the present disclosure also includes methods of diagnosing, monitoring or prognosing melanoma in a host. Embodiments of such methods can include obtaining a sample from a skin lesion of the host or of the host themselves as described above and analyzing the sample with mass spectrometry or other technique to determine a molecular/biomarker signature of the sample. Exemplary methods can also include determining from the molecular/biomarker signature of the sample a level of one or more compounds from a set of melanoma biomarkers.

In embodiments the set of melanoma biomarkers includes, but is not necessarily limited to, the following group of compounds: lactic acid, a compound correlated to the m/z value 86.096, a compound correlated to the m/z value 90.055, benzaldehyde, a compound correlated to the m/z value 109.065, proline, succinic anhydride, betaine, a compound correlated to the m/z value 120.081, benzoic acid, leucine or isoleucine, propylene glycol, histidine, octanoic acid, L-methionine S-oxide, spermidine, methionine, octadienoic acid, 4-hydroxy-L-glutamic acid, phenylalanine, methylhistidine, glycine-proline, aminomuconic acid, phosphocholine, N-acetyl-L-glutamic acid, a compound correlated to the m/z value 188.071, acetyl spermidine, 5-hydroxyindoleacetic acid, a compound correlated to the m/z value 191.089, a compound correlated to the m/z value 192.069, tetrahydrodipicolinate, thiopurine, a compound correlated to the m/z value 204.123, tryptophan, a compound correlated to the m/z value 208.116, carmustine, n-formylmethionine, a compound correlated to the m/z value 225.097, 4-(glutamylamino) butanoate, glutamylalanine, propionyl-L-carnitine, phosphatidylglycerol, 5-(4-Chloro-3-hydroxy-1-butynyl)-2,2′-bithiophene, ethyl glucuronide, aspartyl-gamma-glutamate, aspartyl-glutamine, DOPA sulfate, a compound correlated to the m/z value 288.253, alanine-leucine-proline, diacetyl spermine, a compound correlated to the m/z value 310.235, skullcapflavone I, trihydroxyoctadecenoic acid, adenosine monophosphate, inosine 5′-monophosphate, alanine-glycine-histidine-proline, and the group of lipids corresponding to the group of m/z values consisting of: 560.369, 568.363, 700.530, 750.535, 810.599, 818.727, 854.595, and 879.735. In embodiments, the methods further include comparing the level of the one or more melanoma biomarkers from the host sample with the level of the melanoma biomarkers in a control sample, whereby a significantly different level of the biomarker compound in the test sample as compared to the level of the biomarker compound in the control sample is indicative of melanoma.

Embodiments of the present disclosure also include a system for real-time detection of melanoma in a host. In embodiments, the system includes a device for provoking an emission (e.g., a molecular emission, in embodiments, a volatile, semi-volatile, non- volatile or combination thereof emission) from a skin lesion of a host suspected of including melanoma to produce a molecular sample from the host, such as, but not limited to the methods for provoking a signal described above (e.g., a swab, or other device for abrasion of the lesion; a vacuum aspirator, a DESI device, etc.). In embodiments the sample is a gaseous volatile sample (which may also include semi-volatile compounds, as defined with volatile compounds above). In embodiments, the sample is a lipid sample or other molecular sample that may not be completely gaseous/volatile. In embodiments, the system also includes a sample reservoir for containing or capturing the sample including the emission from the skin lesion, where the sample reservoir is configured to interface with a mass spectrometry device such that the mass spectrometry device is able to analyze the sample. The system also includes a mass spectrometry device adapted to interface with the sample reservoir and capable of analyzing the volatile, semi- and/or non-volatile compound content of the sample. In embodiments, the system also includes a signal processing mechanism in operative communication with the mass spectrometry device, where the signal processing mechanism can also have data transfer and/or evaluation software protocols configured to transform raw data from the mass spectrometry device into information regarding melanoma biomarkers, wherein the information comprises one or more of the presence, absence, and amounts of the melanoma biomarkers. In some embodiments, the signal processing mechanism may be in operative communication with other elements of the system, such as the sample reservoir. In embodiments the signal processing mechanism can provide diagnostic information regarding the melanoma status of the sample (e.g., cancerous vs. benign lesion).

In embodiments the signal processing mechanism can be, but is not limited to, a personal computer, a mainframe, a portable computer, a personal data assistant, a smart phone, and a tablet computer, or a combination thereof. In embodiments, the biomarker detection system is portable and adapted for sampling biomarkers in an office or operating room environment. Other embodiments include a smart phone application configured to receive information from the signal processing mechanism and transform the information into alerts, recommendations, or both for a user. In some embodiments, the computer is a portable personal computer (e.g., “laptop”) that includes data transfer and evaluation software capable of storing and analyzing the recorded signals. Under these circumstances, the melanoma detection system can provide a diagnostic tool that itself is portable and is powered from the laptop computer.

In embodiments, the signal processing mechanism has data transfer and evaluation software protocols configured to transform raw data from the mass spectrometry device into information regarding melanoma biomarkers and other compounds. Examples of such information includes, but is not limited to, the presence, absence, and/or amounts of certain compounds, such as melanoma biomarkers, or a combination of one or more of those. In embodiments, the signal processing mechanism produces a molecular/biomarker signature corresponding to the host sample. In embodiments, the signal processing mechanism compares the molecular signature of the host sample to the molecular signature of a control (cancer-free) sample or to an average of molecular signatures or biomarker amounts from multiple control samples. The results can be further processed by software run by the signal processor to provide diagnostic information or the raw data or molecular signature can be further analyzed by a clinician or other operator.

The system of the present disclosure is capable of providing a specific molecular/biomarker signature of a host sample that is optimized to yield the maximum diagnostic value. These real-time detection systems thus can represent a complete diagnostic package with the capability to aid rapid analysis by a person who has minimum technical training. In exemplary embodiments, the raw data output from the melanoma detection system of the present disclosure is collected and transferred to the memory of a computer, which includes a pattern recognition evaluation program that can be “trained” to identify certain melanoma biomarkers and their amounts and patterns and also the degree of the matching between molecular signature of the host sample and a control molecular signature, and thus recognize the signature of a sample likely to be positive for melanoma as well as a signature of a sample that is “normal” i.e., likely negative for melanoma. In embodiments the system outputs a preliminary diagnosis of melanoma (positive for melanoma) or a negative result/no melanoma based on the comparison of the host sample molecular/biomarker signature and one or more control molecular/biomarker signatures. Such a detection system provides a complete diagnostic package whose purpose is to aid rapid screening, detection, and analysis of a host sample(s), without elaborate preparation or invasive procedures, by a person who has minimum technical training and to enable portability of such a system, bringing heretofore unavailable real-time diagnostic and monitoring capabilities to the clinic/doctor's office. Variations of the methods and systems described above are contemplated within the scope of the present disclosure.

Additional details regarding the methods and compositions of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1% to about 5%, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

### EXAMPLES

### Example 1

### Materials & Methods

### Cell Preparation

SK-MEL-28 and A-375 (male and female malignant melanoma cells, respectively) were grown and maintained in EMEM, while the normal Adult Melanocyte cells were grown and maintained on Dermal Cell Basal Medium supplemented with a melanocyte growth kit (ATCC). Once confluent, cells were washed with H₂O, pelleted by centrifugation, and stored at −20° C. or below until analysis. Upon analysis passages from cells of a given type were pooled by suspension in H₂O. Pellet solutions were vortexed and centrifuged before adding cooled 80% MeOH/H₂O to precipitate out proteins.⁸ Solutions were again vortexed and centrifuged, then the supernatant removed and dried under nitrogen at 35° C. Dried samples were reconstituted with 30 μL 0.1% FA in H₂O for LC/HRMS analysis and 80% MeOH/H₂O for APCI and DART/MS analysis.

### Instrumentation and Chromatography

Data were collected on three instrumental setups:
1) Agilent 6220 TOF MS utilizing DART
2) Thermo Scientific LTQ XL MS employing APCI
3) Thermo Scientific Q Exactive MS applying heated electrospray ionization (HESI) and coupled to LC
Column: ACE Excel 2 C18-PFP, 100×2.1 mm
Pump: Thermo Dionex UltiMate 3000
Parameters: Column temp—35° C., Flow rate—300 μL/min, Injection volume—2 μL
Mobile Phases: Solvent A—0.1% FA/H₂O, Solvent B—CAN

### Data Processing

Multivariate data analysis was performed through MetaboAnalyst⁹ software, in addition to Thermo Scientific's SIEVE. ChemCalc¹⁰ was also employed for high-resolution compound analysis. Several databases were utilized for identification of select m/z values: HMDB, KEGG, LIPID MAPS, and PubChem

### Results and Figure Descriptions

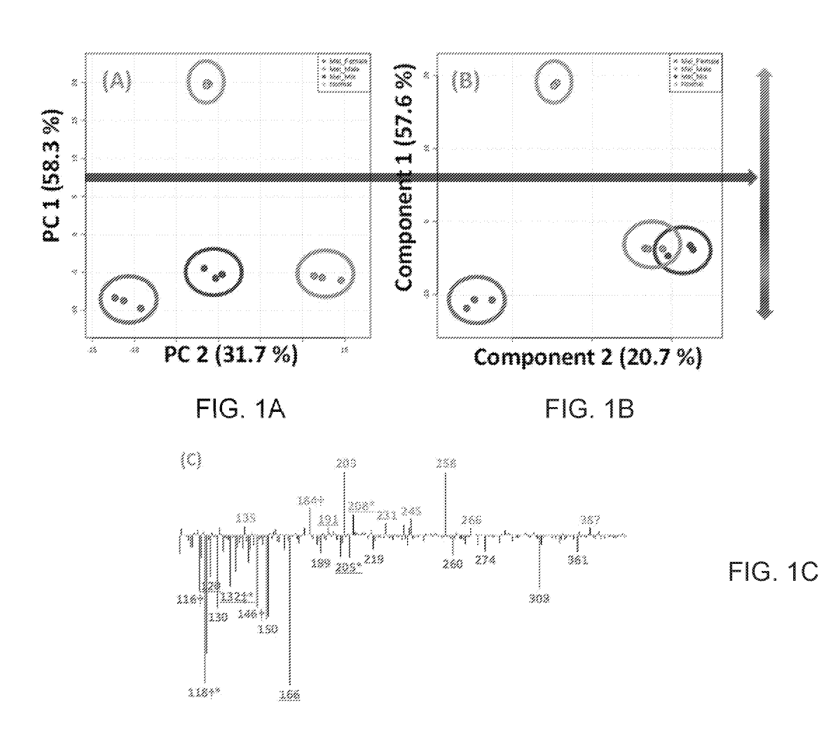

The results of the analysis are illustrated in FIGS. 1-4. FIG. 1A shows the principal component analysis (PCA) of the samples, and FIG. 1B shows the partial least squares discriminant analysis (PLSDA) score plots. FIG. 1C is a PLSDA loadings plot showing first component separation of normal (upper numbers) cells vs. melanoma (lower). Samples from males (M), females (F), pooled males and females (M/F) and normal (N) are indicated. Underlined m/z values in FIG. 1C were also found in a manual inspection of LC data. High-resolution data permits molecular feature extraction, which allowed creation of a table of potential biomarkers for melanoma (Table 1).

TABLE 1
Potential Biomarkers of Melanoma

| | | Proposed | Potential |
|---|---|---|---|
| m/z (± ppm) | | Formula | Compound Name |
| 107.0492¹ | (+0.13) | C₇H₆O₂ | Benzaldehyde |
| 116.0704^{†} | (−0.10) | C₅H₉NO₂ | L-Proline |
| 132.1019* | (−0.22) | C₄H₉N₃O₂ | L-Leucine |
| | | | (or L-Isoleucine) |
| 135.1016 | (−0.17) | C₆H₁₄O₃ | Polypropylene glycol |
| 145.1223¹ | (+0.16) | C₈H₁₆O₂ | Octanoic acid |
| 146.1652^{†} | (−0.19) | C₇H₁₉N₃ | Spermidine |
| 166.0863 | (+0.04) | C₉H₁₁NO₂ | L-Phenylalanine |
| 205.0974 | (+0.11) | C₁₁H₁₂N₂O₂ | L-Trypotophan |

*Denotes m/z value is also present in mass spectrometric analysis of the media in which the cells were grown
^{†}Indicates m/z value is also present in the mass spectra from analysis by at least one of the other methods,
‡Indicates m/z value is also present in the mass spectra from analysis by both of the other methods.

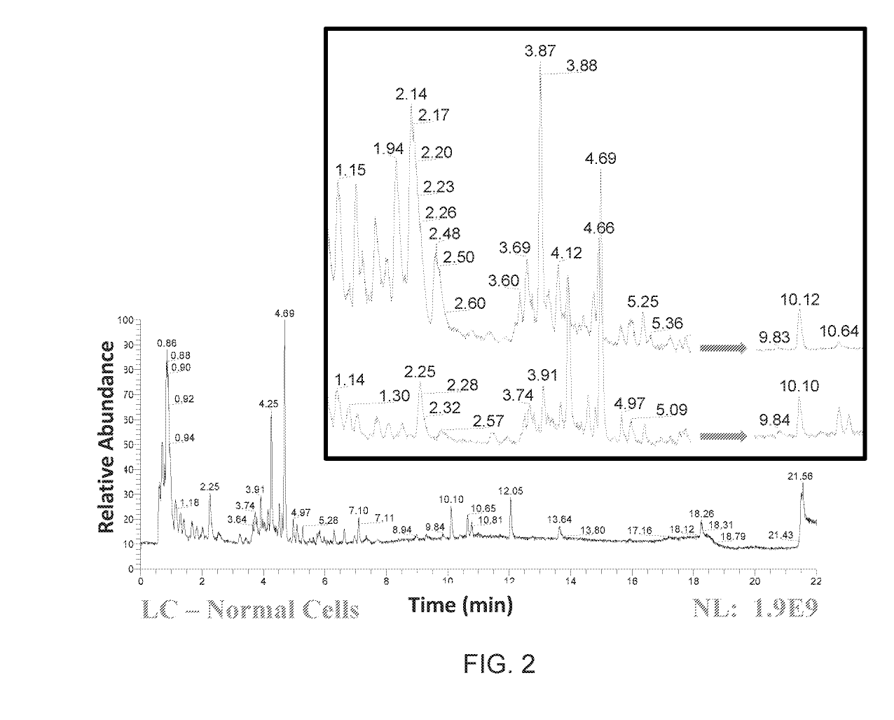

FIG. 2 shows total ion chromatogram (TIC) for LC/HESI/HRMS of normal cells. The inset is a TIC from 1-5.5, 9.5-11 min for normal (lower plot) cells and pooled male/female melanoma (upper plot) cells. Notable differences in r.t. between normal and melanoma species include 1.94-2.25, 3.88, 4.60, 4.69, 10.64, and 10.80. Analysis of these chromatographic peaks reveals melanoma cells demonstrate a change in concentration at the following m/z:

Increase in concentration→86.0964†*, 107.0492¹, 109.0649¹, 120.0808, 132.1019‡*, 145.1223¹, 166.0862/3, 188.0708, 192.0689, 204.1230†, and 205.0974*

Decrease in concentration→191.0894, 208.1159*, 288.2532, 310.2352

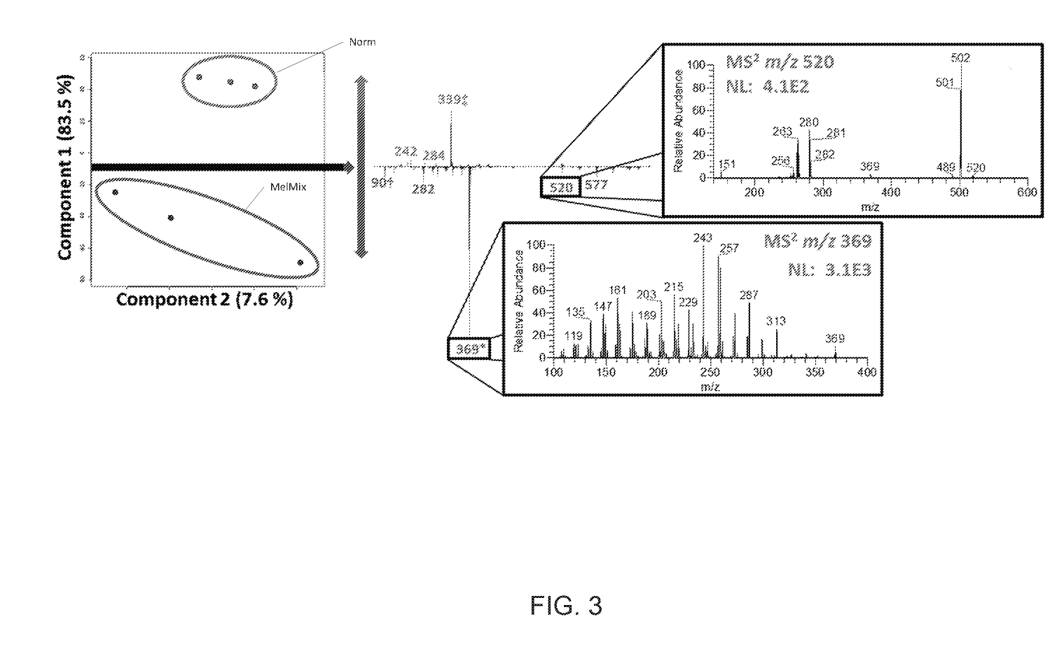

PLSDA score and loadings plots are provided in FIG. 3, showing first component separation of normal cells (upper points/oval) against pooled male/female melanoma (lower points/oval) cells. PLSDA reveals m/z 369 as a statistically significant peak between cell types, yet it is also present in PLSDA of the media. However, it is much more prevalent in the cells, which were washed of media prior to analysis; therefore, it supporting its relevance. Additionally, the increase in intensity of this peak may be due to fragmentation of m/z 520 (upper, right hand plot).

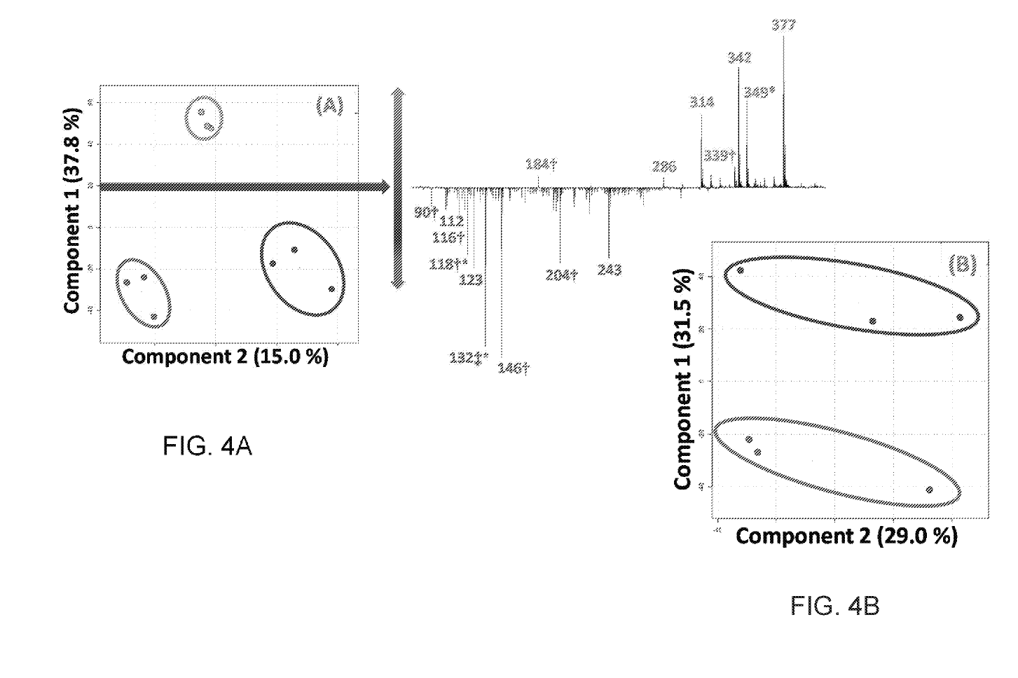

FIGS. 4A-B illustrate PLSDA score and loadings plots showing first component separation of normal cells vs. male and female melanoma cells (FIG. 4A), and PLSDA score plot showing first component separation of male vs. female melanoma cells (FIG. 4B). DART analysis will allow determination of the more volatile species present in melanoma cells.

### Conclusions

The results reveal an apparent difference in the concentration of several species present in melanoma cells as compared to normal human melanocytes, with and without chromatographic separation prior to mass analysis

Currently, a portion of the wealth of information that can be gleaned from the MS data collected has been probed. Further analysis of the data will likely produce additional m/z peaks inherently different between melanoma and normal cells; hence, more potential biomarkers for melanoma may be identified.

PLSDA of the melanoma cells produced a separation by patient sex; however, this may be attributed to differences in the variation of compounds present between individuals rather than solely from male vs female (FIG. 4B). Further studies will involve additional analysis of gender differences (i.e., additional cell lines).

Three ionization techniques used in the present example were compared. All three demonstrated first component separation between melanoma and normal cells by PLSDA.

Probable chemical formulas of potential biomarkers for melanoma were identified by utilization of high-resolution mass spectrometry (Table 1), in addition to an abundance of nominal m/z values isolated in melanoma by multivariate analysis. MSⁿ will be employed for further identification of these m/z values. Utilization of other direct sampling methods, in addition to DART (e.g., LAESI and DESI) will allow identification of other potential biomarkers. The present example demonstrates a method for identification of melanoma with less sample preparation than traditional methods. These methods utilize less solvent and more concentrated samples, resulting in quicker analysis, less cost and less waste. The present methods also allow for analysis of a more native sample environment. Using these techniques for direct analysis of tissue samples can provide a much more analogous metabolome to living skin.

Recent DART/MS analysis of the cellular protein precipitate (from the MeOH-lysed cells), with no preconcentration/derivitization, produced additional peaks, complementary to those produced by the cell solutions. This could signify additional volatile biomarkers, useful for less invasive diagnostic capabilities. Additional studies were conducted as described in Example 2, below.

### Example 2

### Materials & Methods

SK-MEL-28 and A-375 (male and female malignant melanoma cells, respectively) were grown and maintained in Dermal Cell Basal Medium (DCBM) supplemented with a melanocyte growth kit (ATCC), while the normal Adult Melanocyte cells were grown and maintained only on DCBM with kit. Once confluent, cells were pelleted by centrifugation, the media removed, and the pellets stored at −80° C. until analysis. Passages from cells of a given type were washed (×3) and pooled by suspension in 40 mM ammonium formate (AF). On the third wash, solutions were vortexed and 10 μL of solution was taken for protein quantitation. After washing, cells were pelleted via centrifugation, the supernatant discarded, and 10 μL internal standard added. Metabolites were isolated using 2 mL cooled 80% MeOH/H₂O. 10 Solutions were again vortexed and centrifuged, then the supernatant and protein separated. The protein was saved for DART/MS analysis, while the supernatant was dried under nitrogen at 32° C. Dried samples were reconstituted with 30 μL 0.1% FA in H2O for LC/HRMS analysis and 250 μL 80% MeOH/H2O for APCI and DART/MS analysis.

Protein content was quantified by Qubit Protein Assay 11, performed on a Qubit 3.0 Fluorometer. Standards were analyzed by fluorescence spectrometry followed by similar analysis of samples to allow normalization of data.

Data were collected on three instrumental setups: an Agilent 6220 TOF MS utilizing direct analysis in real time (DART), a Thermo Scientific LTQ XL MS using corona-discharge atmospheric-pressure chemical ionization (APCI), and a Thermo Scientific Q Exactive MS applying heated electrospray ionization (HESI) and employing pre-MS separation by liquid chromatography (LC). DART samples were analyzed by either placing the sample directly into the ionization path (tissue) or by dipping a disposable glass pipette into the solution or solid to extract a small amount of analyte (cell culture). The tip of the pipette containing the analyte was then placed into the heated gas stream between the DART source and MS inlet. LC was performed through an ACE Excel 2 C18-PFP, 100×2.1 mm column held at 30° C., utilizing a Thermo Dionex UltiMate 3000 to maintain a flow rate of 350 μL/min and an injection volume of 5 μL. Two mobile phases were employed over a 22 minute separation: Solvent A—0.1% FA/H2O, Solvent B—ACN.

Multivariate data analysis was performed through XCMS package in RStudio¹² and MetaboAnalyst¹³ software. Several databases were utilized for identification of select m/z values, including: HMDB, m/zCloud, and METLIN.¹⁴⁻¹⁶

### Results & Figure Descriptions

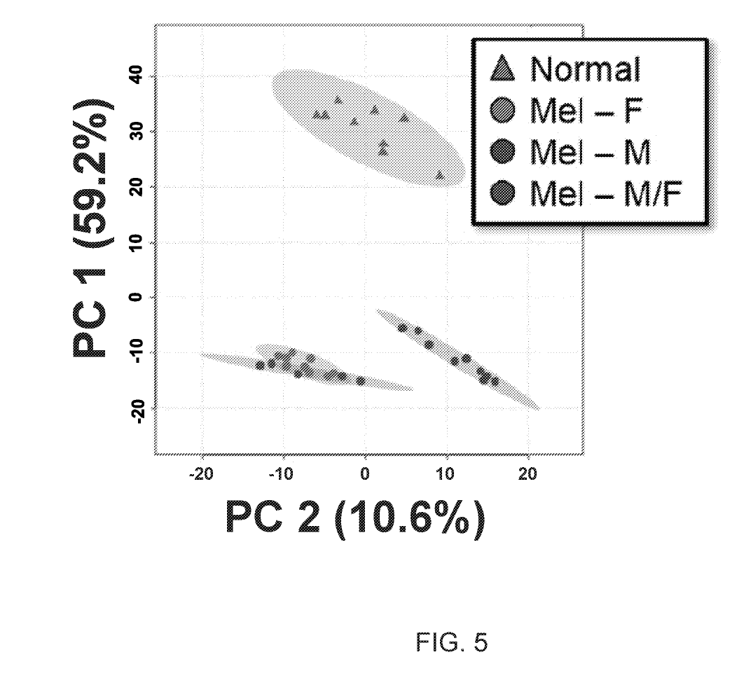

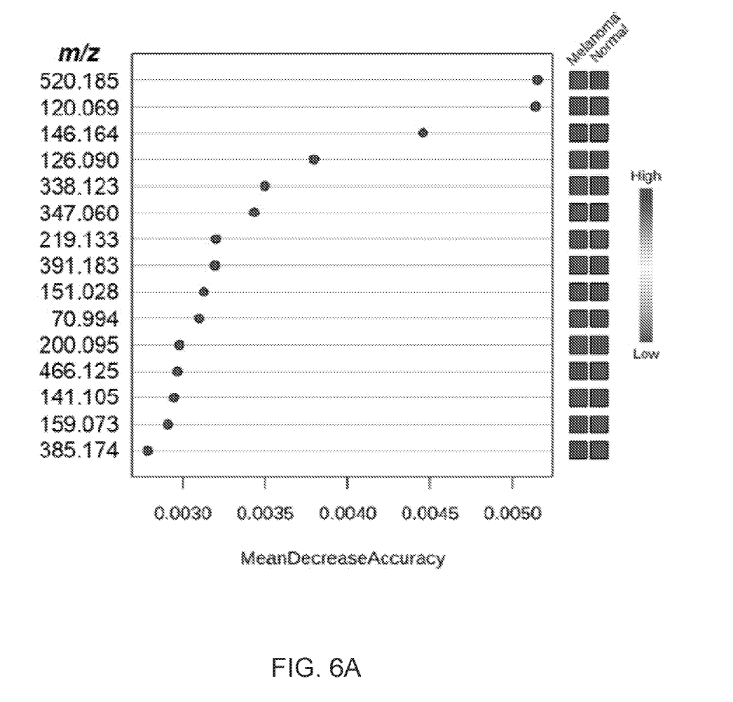

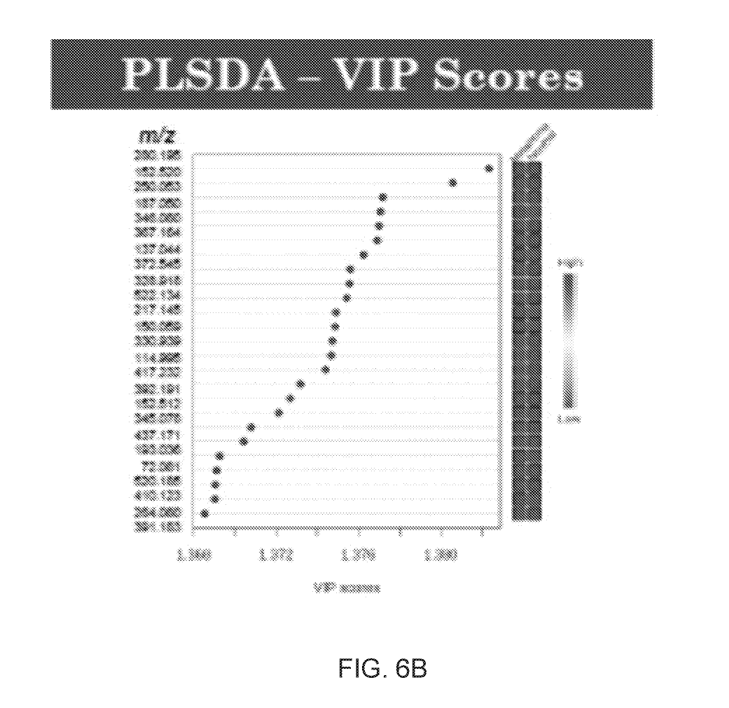

Initially, UHPLC/HR mass spectra were collected for the isolated metabolites of normal adult melanocytes (AN) as well as female and male malignant melanomas (FM and MM, respectively). As the current gold standard, LC/MS allows validation of later direct sampling methods. Furthermore, accurate-mass data permits molecular feature extraction, meaning chemical formula and compound identification of m/z values; hence, table of putative biomarkers may be established. Several multivariate data methods were utilized for data reduction and feature extraction, including principal component (PC) analysis, partial least squares discriminant analysis (PLSDA, FIG. 5), random forest variable classification (RF, FIGS. 6A and 6B). Seven m/z values are shown in Table 2 as prospective biomarkers for melanoma; The top 4 m/z values correspond to a fold change of melanoma/normal greater than one, while the bottom 3 correspond to a fold change of melanoma/normal less than one. These masses were selected based on ppm error, biofunction of the compound, r.t., and polarity. Compound identities were selected to best represent potential biomarkers for melanoma. Of those shown, four are up-regulated in melanoma vs normal samples, while three are down-regulated. A significant finding was that of m/z 166.0863, corresponding to L-phenylalanine. Not only is the ppm error essentially zero, but phenylalanine increases in concentration with the presence of melanoma tumors. Another noteworthy discovery revealed tryptophan (m/z 205.0969) is at a greater concentration in melanoma vs normal samples, whereas aminomuconic acid (m/z 175.0713), a degradation product of tryptophan, shows a significantly lower abundance in melanoma samples. This is interesting as it may suggest that melanoma disrupts this degradation pathway.

FIG. 5 is a PC analysis scores plot displaying first component separation of normal cells (triangles, top oval) vs. melanoma cells (lower ovals). Of the lower, melanoma cells, female (F), male (M), and hybrid male/female (MF) are shown. Accurate-mass data permits molecular feature extraction, allowing creation of a table of potential biomarkers for melanoma (Table 2, below).

FIGS. 6A and 6B illustrate m/z values of various biomarkers. FIG. 6 A is a plot of mean decreased accuracy (MDA) plot of the 15 m/z features from UHPLC/HRMS analysis that contribute most to the random forest training set. FIG. 6B is a plot of the top 25 m/z features from UHPLC/HRMS analysis with the greatest variable information processing (VIP) scores from PLS-DA. The column on the right of each plot indicates whether a given feature is up or down regulated in melanoma vs normal samples.

TABLE 2
Potential Biomarkers of Melanoma

| | Proposed | Potential |
|---|---|---|
| m/z (± ppm) | Formula | Compound Name |
| 118.0864 (+0.85) | C₅H₁₁NO₂ | Betaine |
| 150.0586 (−1.99) | C₅H₁₁NO₂S | L-Methionine |
| 166.0863 (+0.00) | C₉H₁₁NO₂ | L-Phenylalanine |
| 205.0969 (−1.46) | C₁₁H₁₂N₂O₂ | L-Trypotophan |
| 175.0713 (+0.00) | C₆H₇NO₄ | Aminomuconic Acid |
| 194.0422 (−1.03) | C₇H₉NO₄ | Tetrahydrodipicolinate |
| 348.0706 (+0.57) | C₁₀H₁₄N₅O₂P | Adenosine |
| | | Monophosphate |

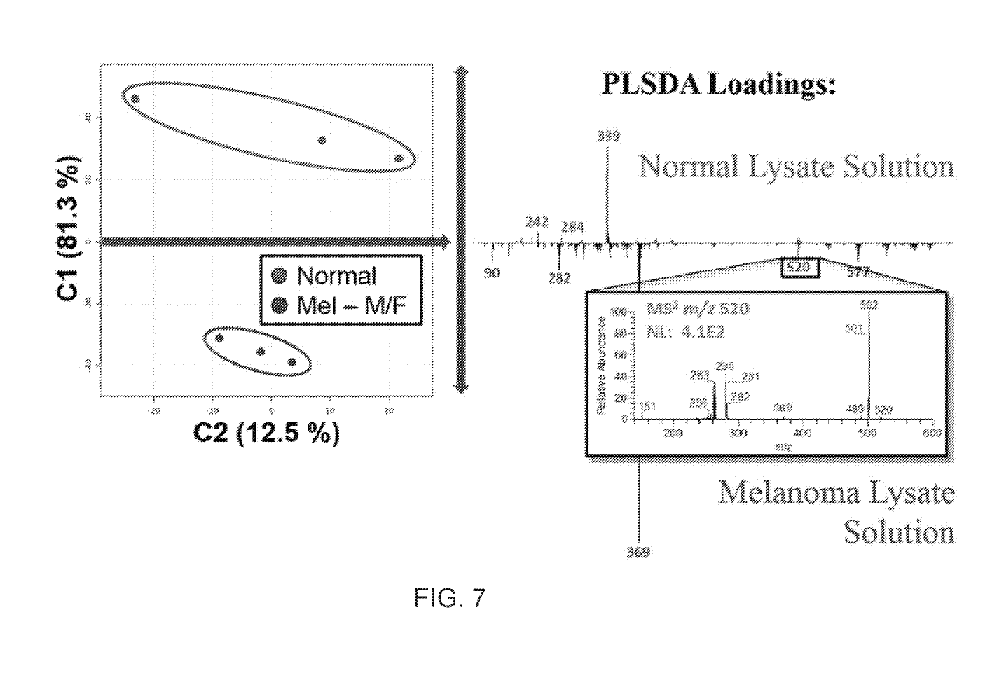

The AN, FM, and MM metabolite isolates were also sampled by APCl/MS to determine if an ambient ionization method could produce meaningful results without the use of a separation step prior to mass analysis. Moreover, the fragmentation analysis capabilities afforded by tandem MS could prove incredibly beneficial. Many of the m/z features extracted from the UHPLC/HRMS data were difficult to identify due to the possibility they were comprised of various combinations of peptides. PCA and PLSDA were again performed to help in identifying m/z peaks of interest between N and MM samples (FIG. 7).

FIG. 7 illustrates PLSDA score and loadings plot showing first component separation of normal cells against pooled male/female melanoma cells.

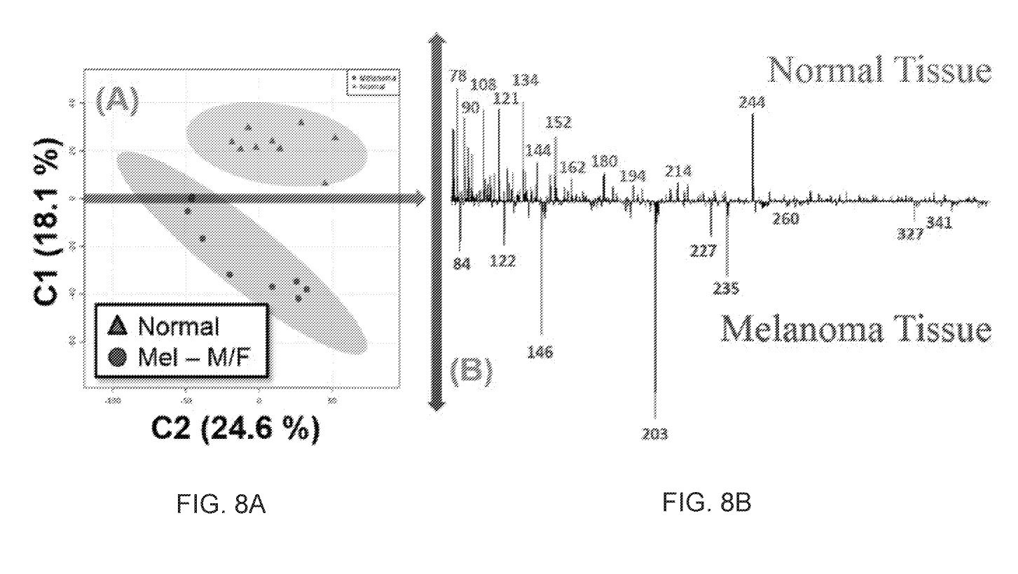

Finally, DART/HRMS was performed on the AN, FM, and MM isolate solutions, in addition to the solid protein lysate, to assess the capabilities of a direct sampling method. Once more, PCA and PLSDA were performed on the data revealing consistent first-component separation. Subsequently, biopsied human tissue samples were obtained and analyzed by DART/HRMS with no prior treatment or extraction. PCA, PLSDA (FIGS. 8A-8B), and RF statistical methods were all applied to the tissue data.

FIGS. 8A-8B illustrates PLSDA scores plot (8A) and loadings plot (8B) showing first component separation of normal vs. male and female melanoma biopsied human skin tissue. As in FIG. 5, the mass accurate data allows molecular feature extraction, providing more potential biomarker compounds for Table 2.

A hierarchical clustering (HCA) was created (not shown) showing branching amongst two groups, normal and melanoma. The top 50 features from UHPLC/HRMS analysis were ranked by t-test and ANOVA statistics to retain the most contrasting pattern (data not shown). Branching was even more defined between female, male, and hybrid male/female melanoma cells.

There are many m/z values which reveal up or down regulation in melanoma vs normal samples. For instance, m/z 90.0548, 118.0864, and 225.0973 are of higher concentration in all three melanoma samples compared to normal samples; m/z: 175.0713, 194.0422, and 348.0706 are of significantly higher concentration in the normal samples vs the melanoma.

### Conclusions

Overall, three different ionization methods were utilized for analysis of cell cultures and human tissue biopsies to determine features of greatest variance between normal and melanoma samples. The results reveal a significant difference between the two sample types, allowing establishment of a table of putative biomarkers for melanoma. For instance, substantial variance was shown between human cell cultures (metabolite and protein content) as well as human skin tissue biopsies. Analysis was performed with and without chromatographic separation prior to mass analysis, in addition to by direct analysis of samples without pre-concentration and/or extraction. PLSDA also demonstrated variation between male/female melanoma.

Further probing of the data should continue to yield more compounds as potential biomarkers of melanoma, as well as eventual quantification of biomarker concentrations. Ongoing experiments incorporate analysis of swabs of human skin by DART/MS. Additionally, DESI and LAESI can be utilized in the future to allow localization of cancerous cells/tissue in a given sample.

### Example 3

### Global LC-HRMS Metabolomic Analysis of Novel Melanoma Skin Tissue Biomarkers Employing Positive and Negative Ionization Modes.

The use of biomarkers to aid in the early detection of cancer is a priority in cancer-related research and clinical care. Earlier identification of cancers such as melanoma increases the likelihood of survival and successful recovery. While many studies in the literature have been devoted to the genomic and proteomic analysis of melanoma skin cancer, few have assessed the metabolome and lipidome of melanoma for biomarker discovery. This Example investigates the metabolome of normal and melanoma skin tissues using LC-HRMS and MS/MS methodologies to identify additional melanoma biomarkers and better understand the disease etiology. In addition, by analyzing metabolites in a more native environment, this will allow for the translation of disease discovery in a clinical setting.

### Methods

Melanoma and normal skin tissue samples were normalized to wet weight (50 mg), homogenized in methanol (550 μL) using 3 mm borosilicate and 0.7 mm zirconia beads, and extracted using the Folch lipid extraction methodology. The aqueous layer was collected, dried down under nitrogen, and reconstituted in 200 μL of 0.1% formic acid in water. Protein concentrations were obtained using a Quibit Protein Assay using 5 μL of the homogenized tissue sample. The organic layer of the samples was dried and reconstituted in 200 μL of isopropanol.

Instrumentation and Chromatography

Data were collected on a Thermo Scientific Dionex UltiMate 3000 RS UHPLC pump coupled to a Thermo Scientific Q Exactive orbitrap mass spectrometer. Samples were analyzed in triplicate. Data collected on a Thermo Scientific Q Exactive employed polarity switching in positive and negative ion mode. Tandem MS (data-dependent top 10, ddMS² and all ion fragmentation, AIF) analysis were incorporated in positive and negative ion mode for the identification of endogenous lipid species and only Top10-ddMS² was applied for the metabolites. Samples were normalized to the corresponding protein concentrations post-data acquisition.

### Metabolites

Column: ACE Excel 2 C18-PFP column (100×2.1 mm, 2 μm)
Parameters: Column temp—35° C., Flow rate—350 μL/min, Injection volume—5 μL
Mobile Phases: Solvent A: 0.1% formic acid water, Solvent B: CAN
Gradient: 0-3 min. hold at 0% B, a linear ramp to 80% B at 13 min, a hold at 80% B until 16 min

### Lipids

Column: Supelco Analytical Titan C18 column (75×2.1 mm, 1.9 μm)
Parameters: Column temp—30° C., Flow rate—500 μL/min, Injection volume—2 μL
Mobile Phases: Solvent C: 60:40 acetonitrile/water with 10 mM ammonium formate and 0.1% formic acid, Solvent D: 90:8:2 isopropanol/acetonitrile/water with 10 mM ammonium formate and 0.1% formic acid
Gradient: 0-1 min. hold at 0% B, a linear ramp to 65% B at 11 min, a hold at 65% B until 13 min, a linear ramp to 95% B at 18 min, and a hold at 95% B until 20 min

TABLE 3
Mass spectrometric instrument parameters for metabolite and lipid
fragmentation using Top10-ddMS² and AIF acquisition modes.
Parameters include m/z range, resolution, automatic gain control (AGC),
injection time (IT), normalized collision energy (NCE), isolation width
(Iso. Width), underfill, intensity threshold (Inten. Threshold) apex trigger,
dynamic exclusion (Dyn. Exclusion). The full scan resolution was reduced
to 35,000 with polarity switching for both metabolites and lipids.

| | Metabolites | | | Lipids | | | |
|---|---|---|---|---|---|---|---|
| Instrumental | Polarity | Full | Top10- | Polarity | Full | Top10- | |
| Parameters | Switching | Scan | ddMS² | Switching | Scan | ddMS² | AIF |
| m/z Range | 70-1000 | 70-1000 | 70-1000 | 100-1000 | 70-1000 | 100-1000 | 100-1000 |
| Resolution | 35,000 | 70,000 | 17,500 | 35,000 | 70,000 | 17,500 | 70,000 |
| AGC | 3 × 10⁶ | 5 × 10⁶ | 5 × 10⁶ | 3 × 10⁶ | 5 × 10⁶ | 5 × 10⁶ | 5 × 10⁶ |
| IT (ms) | 200 | 256 | 100 | 200 | 256 | 100 | 256 |
| NCE | | | 20 ± 5 | | | 20 ± 5 | 20 ± 5 |
| Iso. Width (m/z) | | | 1 | | | 1 | |
| Underfill (%) | | | 0.1 | | | 0.1 | |
| Inten. | | | 5 × 10⁴ | | | 5 × 10⁴ | |
| Threshold | | | | | | | |
| Apex Trigger(s) | | | 5-20 | | | 5-20 | |
| Dyn. | | | 6 | | | 6 | |
| Exclusion(s) | | | | | | | |

Data Processing

All LC-HRMS data were initially processed by the Xcalibur Workstation software (version 3.0). Data processing was further performed using MZmine 2.17 and MetaboAnalyst 3.0 (online). Samples were normalized post-data acquisition to the corresponding protein concentrations using the data output file from MZmine. The following parameters were applied to the metabolite and lipid datasets in MetaboAnalyst: interquantile range filtering, normalization by sum, log transformation, and autoscaling. Exogenous metabolite and lipid internal standards were spiked into the reconstitution solvent to monitor instrument variability and within-run reproducibility. The CV throughout the 22 hour sequence for retention time of the metabolites was less than 0.8%, and the peak area CV was less than 8%. The % CV throughout the lipid sequence for retention time was <8%.

Compound Identification

For metabolites, the Thermo Scientific Compound Discoverer software with the built-in metabolomics workflow (Untargeted Metabolomics Workflow with ID) was used for all Top10-ddMS² files. For lipids, the Thermo Scientific LipidSearch software was used for Top10-ddMS² files and in-house LipidMatch software designed by Jeremy Koelmel was used for AIF and Top10-ddMS² files. For the purpose of this work, only A and B grade LipidSearch ID assignments were used for lipid ID confirmation. In addition, a database search was conducted for the identification of unknowns. The mass tolerance window was constrained to 5 ppm for positive and negative ion mode. Databases utilized included Metlin, Human Metabolome Database (HMDB), LipidMAPS, and KEGG.

### Results and Discussion

The significance analysis of microarray (SAM) is a high-dimensional feature selection method that addresses issues that may result from applying multiple tests to metabolomic datasets by providing a False Discovery Rate (FDR). The delta value is related to the FDR and the number of significant features found in the dataset. An increase in the delta value results in a decrease in the FDR. The Delta plot visualizes the number of metabolites for a given set of delta values and the estimated FDR.

### Metabolites

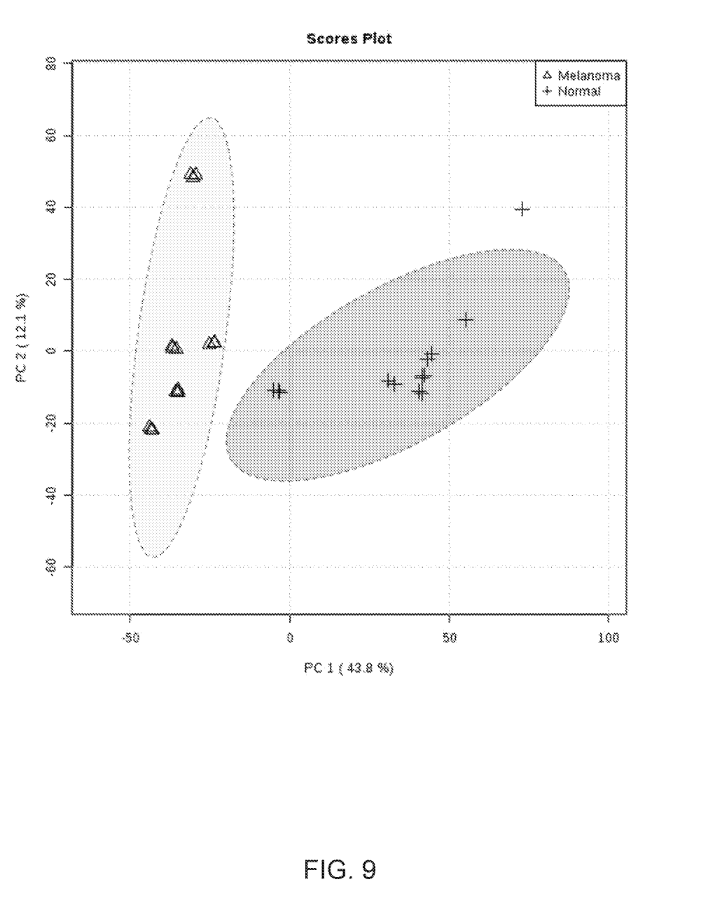

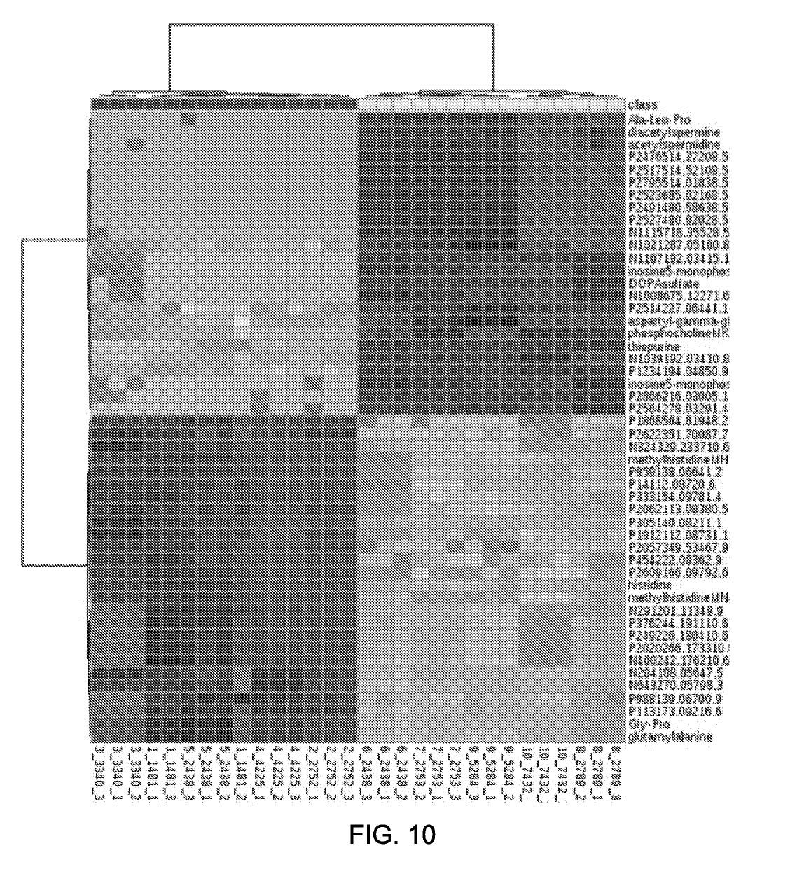

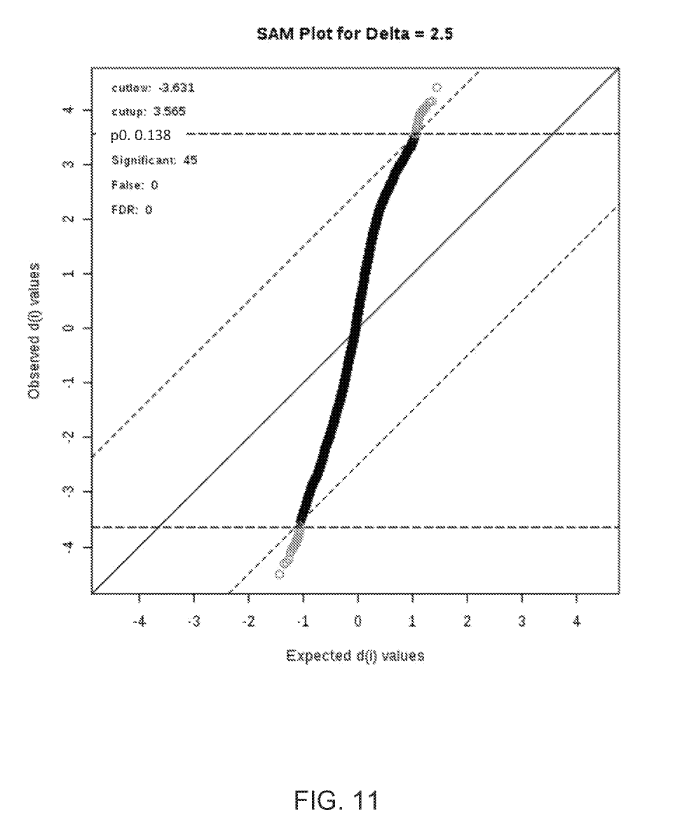

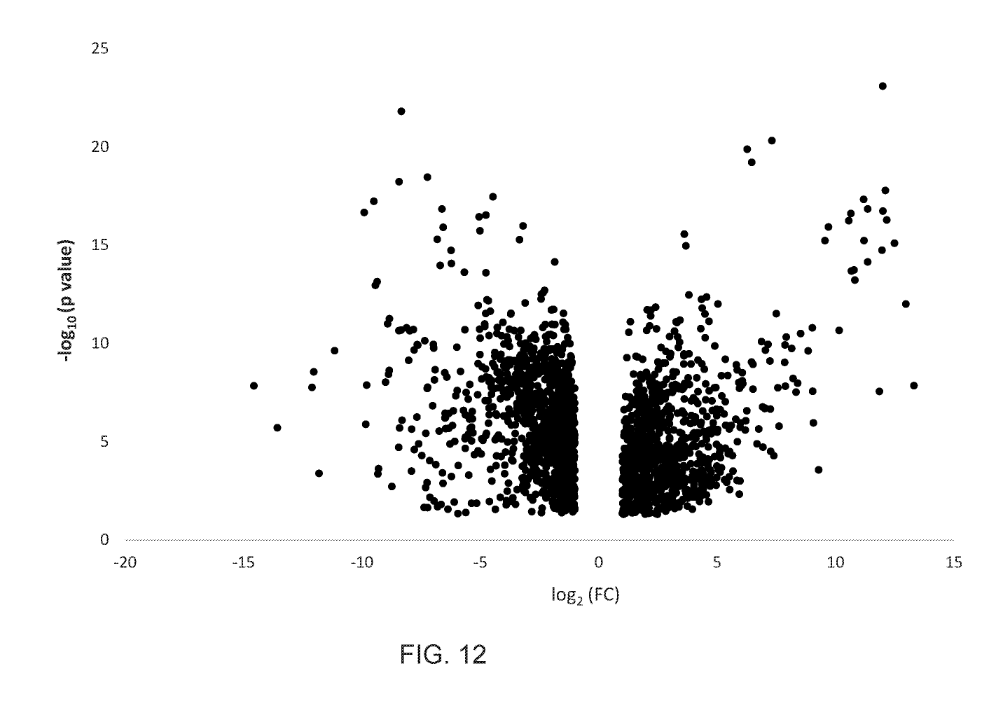

The PCA scores plot (FIG. 9) shows separation between melanoma and the control tissue samples within the first principal component. The top 50 metabolites contributing to the loadings in the positive (melanoma) and the negative (control) directions were selected for putative identification. The top 50 features from the PLS-DA variable importance in projection (VIP) score plot having a VIP score in the first component of 1.0 and higher (data not shown) were added to the list of compounds for univariate analysis and compound identification. Furthermore, the top 200 features from hierarchical clustering plots using random forest (data not shown) and PLS-DA (FIG. 10) were also added to the list for compound identification. The SAM plot (FIG. 11) shows that 45 metabolite features fall within the positive and negative standard deviation distribution estimated by random permutations of the dataset. The FDR rate reported is low for these 45 metabolites indicating that they accurately define the differences between the melanoma and control tissue samples. Random Forest (RF) values were added for all features in the consolidated list. Features were reduced to include compounds having a p value <0.05 and a log₂ (fold change) greater than 1.5. A volcano plot of the data is shown in FIG. 12.

Compound m/z values of interest obtained from the PCA and PLS-DA loading plots that are higher in the melanoma skin tissues include m/z 122, m/z 203, and m/z 341. Compound m/z values of interest obtained from the PCA and PLS-DA loading plots that are higher in the normal skin tissues include m/z 144, m/z 180, and m/z 244.

Table 4 below provides a reduced feature list containing the most significant features with putative identifications from PCA (top 100), PLS-DA (top 50), random forest (top 50), HCA (PLS-DA and random forest- top 100), listed in order of fold change. Features with a higher log2 fold change for the normal are shown in gray and the melanoma is shown in white.

TABLE 4

| | | | | | | | | | | | Mass |
|---|---|---|---|---|---|---|---|---|---|---|---|
| | Feature | RT | | | | | Random | Log₂ | | | Error |
| Polarity | m/z | (min) | PCA | HCA-PLS | HCA-RF | PLS-DA | Forest | FC | Compound Name | Adduct | (ppm) |
| P | 170.093 | 3.5 | | | X | 1.57 | 1.0E−03 | −9.90 | methylhistidine | [M + H]⁺ | −1.18 |
| P | 192.075 | 3.6 | | X | X | 1.58 | 2.2E−03 | −9.50 | methylhistidine | [M + Na]⁻ | −2.08 |
| P | 138.066 | 3.4 | | X | X | 1.61 | 3.2E−03 | −8.33 | histidine | [M + H − H₂O]⁺ | 3.62 |
| N | 239.065 | 6.6 | | | X | 1.57 | 3.6E−04 | −5.05 | glutamylalanine | [M + Na − 2H]⁻ | 0.42 |
| N | 171.078 | 6.6 | | | X | 1.57 | 1.2E−03 | −4.77 | Gly-Pro | [M − H^{[-} | −1.17 |
| P | 164.056 | 2.2 | | X | X | 1.50 | 1.3E−03 | −4.66 | 4-Hydroxy-L-glutamic acid | [M + H]⁺ | −1.22 |
| N | 188.056 | 7.5 | | X | X | 1.58 | 1.6E−03 | −4.47 | N-acetyl-L-glutamic acid | [M − H]⁻ | 0.00 |
| N | 313.071 | 12.4 | | | X | 1.47 | 6.7E−04 | −4.28 | skullcapflavone I | [M − H]⁻ | 1.28 |
| P | 118.050 | 2.2 | X | | X | 1.48 | 1.0E−03 | −4.05 | succinic anhydride | [M + NH₄]⁻ | −0.85 |
| N | 329.234 | 10.6 | | X | X | 1.56 | 1.8E−03 | −3.34 | trihydroxyoctadecenoic | [M − H]⁻ | −1.21 |
| | | | | | | | | | acid | | |
| N | 190.051 | 9.8 | | X | | 1.37 | 2.1E−03 | −3.02 | 5-Hydroxyindoleacetic | [M − H]⁻ | 0.00 |
| | | | | | | | | | acid | | |
| P | 158.118 | 0.9 | | X | | 1.40 | 2.2E−03 | −2.65 | octadienoic acid | [M + NH₄]⁻ | −1.26 |
| N | 248.960 | 0.6 | X | X | | 1.42 | 1.5E−03 | −2.37 | 5-(4-Chloro-3-hydroxy-1- | [M − H₂O − H^{]-} | −2.01 |
| | | | | | | | | | butynyl)-2,2′-bithiophene | | |
| N | 259.023 | 11.4 | | X | X | 1.47 | 1.6E−03 | −1.89 | ethyl glucuronide | [M + K − 2H]⁻ | −0.39 |
| N | 361.164 | 12.2 | X | | | 1.40 | 1.2E−03 | −1.81 | Ala-Gly-His-Pro | [M − H₂O − H]⁻ | −4.71 |
| N | 121.030 | 11.4 | X | | | 1.44 | 6.2E−04 | −1.74 | benzoic acid | [M − H]⁻ | −1.65 |
| P | 186.176 | 2.2 | | | X | 1.57 | 8.3E−04 | 12.17 | acetylspermidine | [M + H]⁺ | 0.00 |
| P | 309.225 | 2.2 | | | X | 1.61 | 8.0E−04 | 12.00 | diacetylspermine | [M + Na]⁺ | 1.94 |
| N | 347.039 | 1.9 | X | | X | 1.52 | 4.4E−04 | 10.82 | inosine 5′-monophosphate | [M − H]⁻ | 1.44 |
| | | | | | | | | | (IMP) | | |
| P | 349.055 | 1.8 | | | X | 1.53 | 4.0E−04 | 10.67 | inosine 5′-monophosphate | [M + H]⁺ | −2.29 |
| | | | | | | | | | (IMP) | | |
| P | 300.191 | 7.1 | | X | X | 1.56 | 1.8E−03 | 9.70 | Ala-Leu-Pro | [M + H]⁺ | 2.33 |
| P | 278.033 | 1.4 | | | X | 1.56 | 6.7E−04 | 9.58 | DOPA sulfate | [M + H]⁺ | 0.00 |
| P | 194.049 | 5.1 | | | X | 1.60 | 1.2E−03 | 7.31 | thiopurine | [M + ACN + H]⁺ | 0.52 |
| P | 214.015 | 1.0 | | X | | 1.08 | 2.4E−03 | 4.62 | carmusline | [M + H]⁺ | 0.00 |
| P | 219.080 | 1.5 | | | X | 1.49 | 7.3E−04 | 4.37 | N-formylmethionine | [M + ACN + H]⁺ | 1.32 |
| N | 260.089 | 0.8 | | X | X | 1.51 | 3.1E−03 | 3.81 | aspartyl-gamma- | [M − H]⁻ | −1.15 |
| | | | | | | | | | glutamate | | |
| P | 222.030 | 0.7 | | | X | 1.56 | 7.3E−04 | 3.62 | phosphocholine | [M + K]⁺ | −1.35 |
| N | 231.098 | 1.1 | | X | X | 1.48 | 1.8E−03 | 3.27 | 4-(glutamylamino)butanoate | [M − H]⁻ | 1.30 |
| N | 262.104 | 0.8 | | X | | 1.41 | 1.9E−03 | 2.71 | aspartyl-glutamine | [M + H]⁺ | −1.53 |
| N | 228.063 | 0.8 | | X | | 1.32 | 2.1E−03 | 2.64 | phosphocholine | [M + FA − H]⁻ | 3.95 |
| N | 245.043 | 0.7 | X | | | 1.41 | 1.0E−03 | 2.52 | phosphatidylglycerol | [M − H]⁻ | 0.41 |
| P | 184.073 | 0.7 | X | | X | 1.49 | 8.0E−04 | 2.05 | phosphocholine | [M + H]⁻ | 0.54 |
| N | 146.028 | 1.2 | | X | | 1.12 | 1.5E−03 | 2.00 | L-methionine S-oxide | [M − H₂O − H]⁻ | −4.11 |
| P | 240.121 | 6.6 | | X | | 1.41 | 2.9E−03 | 1.86 | propionyl-L-carnitine | [M + Na]⁺ | 0.42 |
| N | 71.013 | 1.2 | | X | | 1.34 | 1.4E−03 | 1.85 | lactic acid | [M − H₂O − H]⁻ | 0.00 |

Metabolites from the reduced table that were higher in the control samples included 5-(4-Chloro-3-hydroxy-1-butynyl)-2,2′-bithiophene, amino acid peptides, N-acetyl-glutamic acid, glutamylalanine, trihydroxyoctadecenoic acid, histidine, and methylhistidine.

Biothiophenes have been shown to to be biologically important precursors with antimutagenic/anticancer activity and to have been targeted as therapeutic agents in anticancer studies. Although N-acetyl-glutamate does not have any connection to melanoma cancer in literature, this metabolite is an allosteric activator of the first enzyme involved in the urea cycle. Lower levels of glutamylalanine were found in sun-exposed skin samples compared to the control sun-protected skin samples. These findings suggested that activity within pathways involving y-glutamylaminoacids are reduced/perturbed with unprotected exposure to the sun or the progression of melanoma skin cancer. The conversion of histidine to histamine is regulated by the histidine decarboxylase (HDC) enzyme. Increased levels of histamine lead to immunosuppression and an increased rate of proliferation of malignant growth. Literature has shown melanoma cell lines to have HDC expression that is one tenth of that from normal basophilic cell lines. Urine samples from patients with melanoma have shown methyl histidine to be three times higher than the normal group. This work shows methyl histidine to have a log₂ fold change of 9 in the normal group compared to melanoma tissue samples.⁵

Higher levels of the following metabolites were found in the melanoma tissue samples: inosine monophosphate, thiopurine, phosphocholine, diacetylspermine, and acetylspermidine Malignant melanoma cell studies have highlighted the connection between guanosine monophosphate reductase (GMPR), inosine monophosphate (IMP) and melanoma cell growth. GMPR is an enzyme involved in the reduction of GMP from IMP. Increased GMPR activity has been shown to suppress melanoma cell invasion and growth. Therefore, this work has shown an increase in IMP levels for melanoma tissue samples (log₂ fold change of 10) indicating that GMPR activity is increased. Many studies have highlighted the correlation between melanoma skin cancer risk and inflammatory bowel disease (IBD). The increased use of thiopurine medications for patients with IBD leads to immunosuppressive activity, an increased incidence of melanoma skin cancer in IBD patients, and an increase risk of non-melanoma skin cancer in ulcerative colitis patients. Non-treated melanoma cancer patients show an increase in phosphocholine and a high correlation of this metabolite with tumor growth. A significant positive correlation is observed between diacetylspermine levels and the progression of tumor invasiveness in melanoma skin cancer. This work shows a log₂ fold change of 12 for diacetylspermine in melanoma tissue compared to normal skin tissue. Although acetylspermidine has not been associated with skin cancer before in literature, this work shows the same fold change for this metabolite as diacetylspermine in melanoma tissue compared to the control. Literature has shown increased urine levels of acetylspermidine to be an indicator for the presence of tumors.

### Example 4

### Lipidomics Analysis of Human Melanoma Skin Tissues Implementing In-House Lipid Fragment Libraries for Identification, LC-MS, and Ambient Ionization Methods.

Melanoma is an aggressive skin cancer that has a good prognosis when detected early; unfortunately, early recognition of melanoma and histological diagnosis can be difficult. Identification of biomarkers could improve the early diagnosis of melanoma before lethal stages develop. Many studies explore genomic and proteomic aspects of melanoma, but few have assessed the metabolome and lipidome for biomarker discovery. This work explored the lipid profiles of normal and melanoma skin tissues using UHPLC-HRMS and MS/MS methodologies, as well as the use of ambient ionization for improved throughput. Lipid identification used both an in-house fragmentation library and commercially available software. The biochemical profiles of the putative biomarkers were further explored to better understand the pathology of melanoma.

### Methods

The methodology for LC-HRMS analysis is as described above in Example 3. Briefly, for UHPLC-MS analysis of melanoma and normal skin, tissue samples were normalized to wet weight (50 mg), homogenized in methanol (550 μL) using 3 mm borosilicate and 0.7 mm zirconia beads, and a Folch lipid extraction was used. The organic layer of the samples was dried and reconstituted in 200 ρL of isopropanol. Data were collected on a Thermo Scientific Dionex UltiMate 3000 RSLC coupled to a Q Exactive. Lipid extracts were separated on a Supelco Analytical Titan C18 column (75×2.1 mm, 1.9 μm).

For ambient-MS analysis, a liquid micro-junction probe ESI source was used. Approximately 5mg of melanoma or normal tissues were placed on microscope slides for analysis. Solvents were optimized for lipids in skin tissue.

Data were collected with a liquid micro-junction probe ESI source directly off of the biopsied tissue samples and injected into a Thermo Scientific Q Exactive orbitrap mass spectrometer. Samples were analyzed in triplicate. Data collected on a Thermo Scientific Q Exactive employed polarity switching in positive and negative ion mode. Tandem MS were incorporated in for further identification and validation of compounds.

Data processing was further performed using MetaboAnalyst 3.0 (online). The following parameters were applied to the datasets in MetaboAnalyst: interquantile range filtering, normalization by sum, log transformation, and autoscaling.

The Thermo Scientific LipidSearch software was used for Top10-ddMS² files and in-house LipidMatch software designed by Jeremy Koelmel was used for AIF and Top10-ddMS² files. For the purpose of this work, only A and B grade LipidSearch ID assignments were used for lipid ID confirmation. In addition, a database search was conducted for the identification of unknowns. The mass tolerance window was constrained to 5 ppm for positive and negative ion mode. Databases utilized included Metlin, Human Metabolome Database (HMDB), LipidMAPS, and KEGG.

### Results and Discussion

Preliminary studies employed a metabolomics analysis of melanoma and healthy tissue using Direct Analysis in Real Time-mass spectrometry (DART-MS). DART-MS provided a quick method to analyze whole skin tissues at ambient conditions with limited sample preparation and provided principal component separation between melanoma and normal tissue; however, DART-MS did not provide complete lipid coverage. Few studies in the literature provide a comprehensive analysis of melanoma tissues. This work employed an untargeted lipidomics methodology to identify putative biomarkers for melanoma and the tested the use of a liquid micro-junction probe with ESI as a means to reduce sample preparation and analysis time.

All UHPLC-HRMS data were initially processed by the Xcalibur Workstation software (version 3.0). Data processing was further performed using MZmine 2.17, MetaboAnalyst 3.0 (online), LipidMatch (in-house software), and LipidSearch. Exogenous lipid internal standards were spiked into the samples before extraction to monitor instrument variability and within-run reproducibility. The % CV throughout the sequence for retention time was <8%.

Univariate (e.g., Student t-test, fold change analysis, volcano plots, and significance analysis of microarrays), and multivariate statistical analysis (e.g., PCA, PLS-DA, hierarchical clustering, and random forest) were employed to identify features that differentiate the melanoma and normal skin tissues in LC-MS. The identification of lipids was focused on significant features from the Student t-test (1,344 m/z values have a p-value less than 0.05).

The UHPLC/MS data showed principal component separation between melanoma and normal skin tissue lipid extracts with principal component analysis (PCA) and partial least square-discriminant analysis (PLS-DA). Ions with m/z values 854.595, 750.535, 700.530, and 568.363 were increased in the PCA loadings for melanoma; in contrast ions with m/z values 879.735, 810.599, 560.369, and 818.727 were higher for normal tissue.

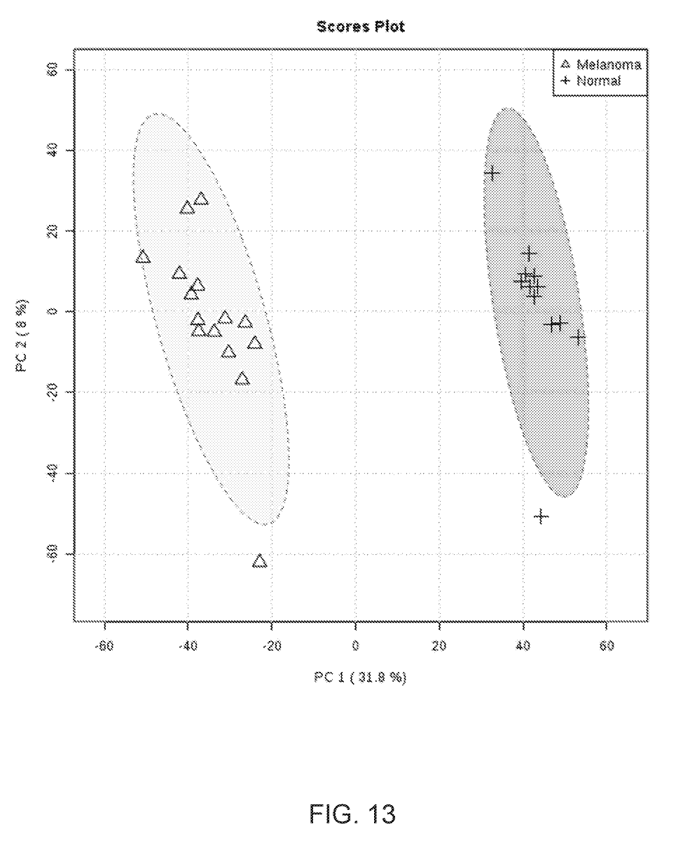

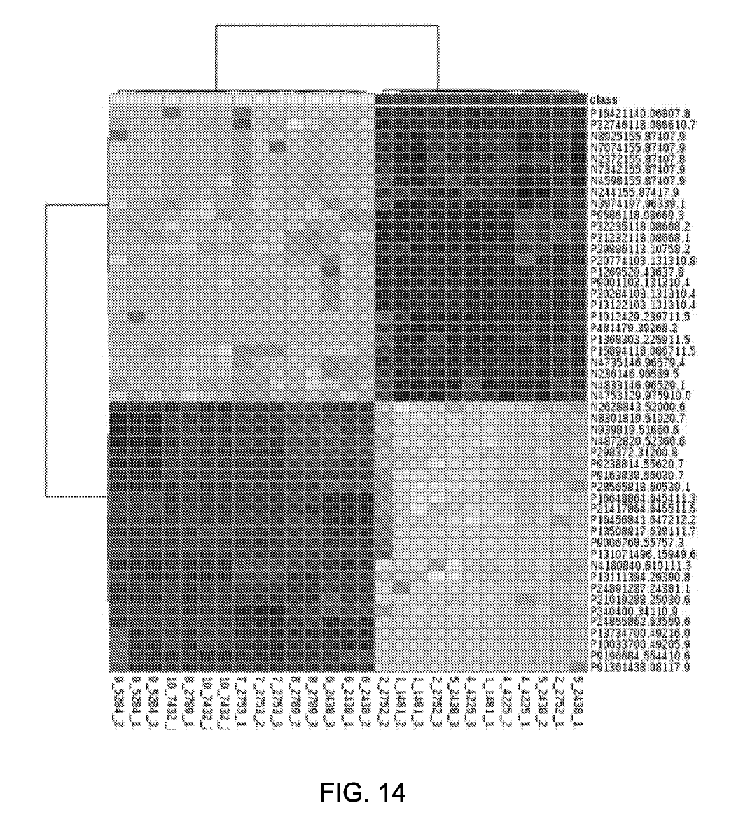

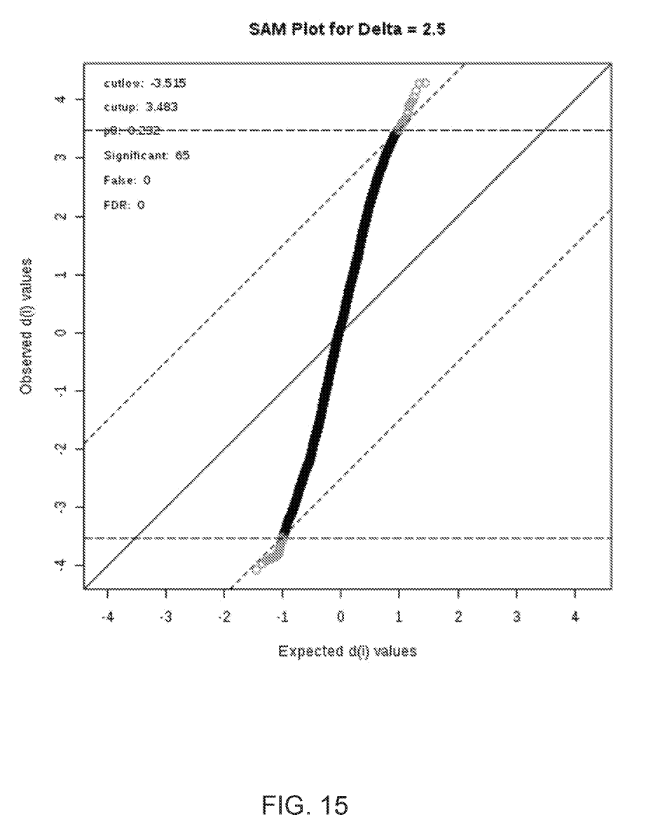

The PCA scores plot (FIG. 13) showed separation between melanoma and the control tissue samples within the first principal component. However, the variance percentage in the first principal component was higher for the metabolites (43.8%) as opposed to the lipids (36.6%). The top 50 lipids contributing to the loadings in the positive (melanoma) and the negative (control) directions were selected for putative identification. The top 50 features from the PLS-DA variable importance in projection (VIP) score plot (data not shown) having a VIP score in the first component of 1.0 and higher were added to the list of compounds for univariate analysis and compound identification. Furthermore, the top 75 features from hierarchical clustering plots using random forest (data not shown) and PLS-DA (FIG. 14) were also added to the list for compound identification. The SAM plot (FIG. 15) shows that 65 metabolite features fall within the positive and negative standard deviation distribution estimated by random permutations of the dataset. The FDR rate reported is low for these 65 metabolites indicating that they accurately define the differences between the melanoma and control tissue samples. Random Forest (RF) values were added for all features in the consolidated list. Features were reduced to include compounds having a p value <0.05 and a log₂ (fold change) greater than 1.5.

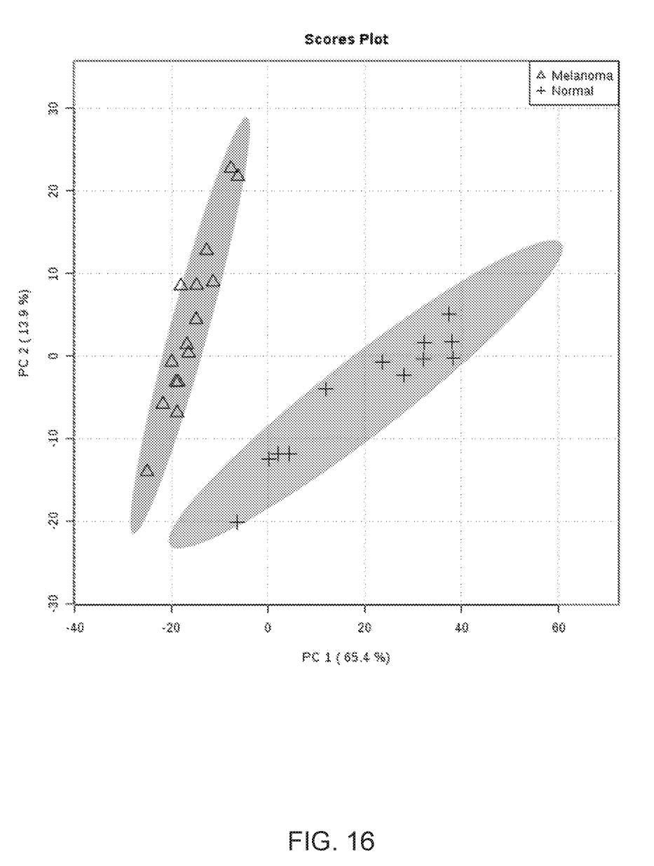

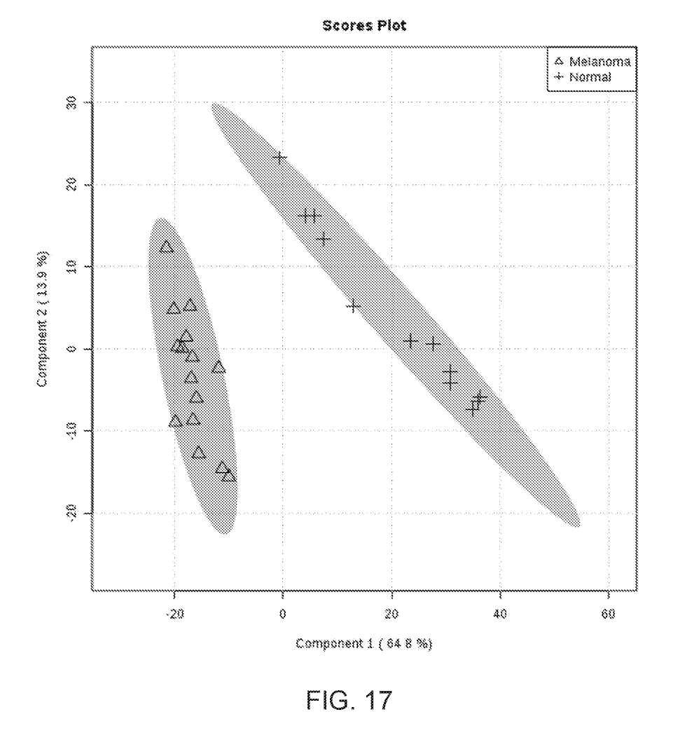

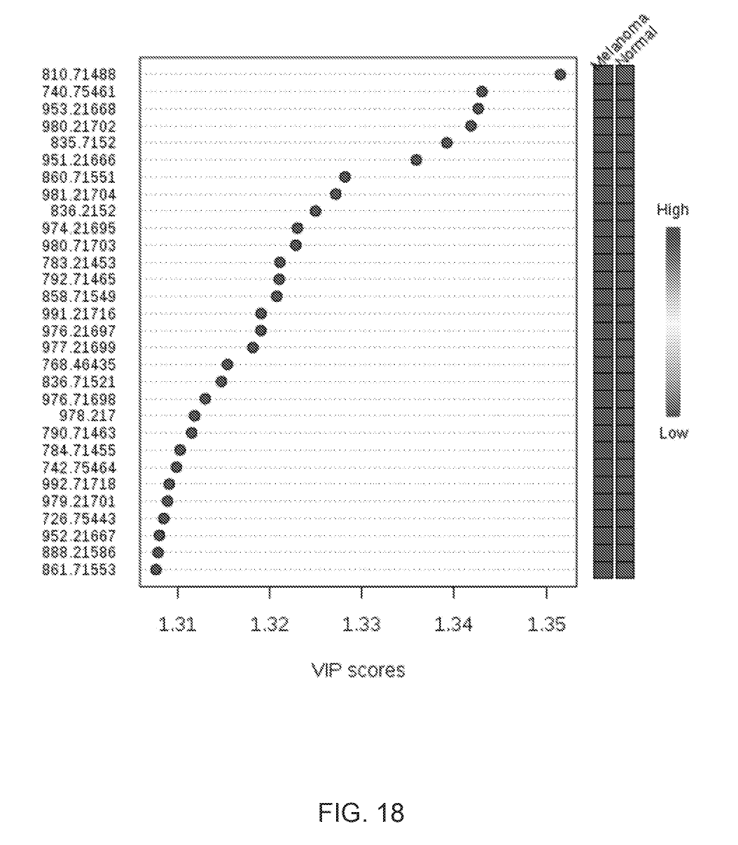

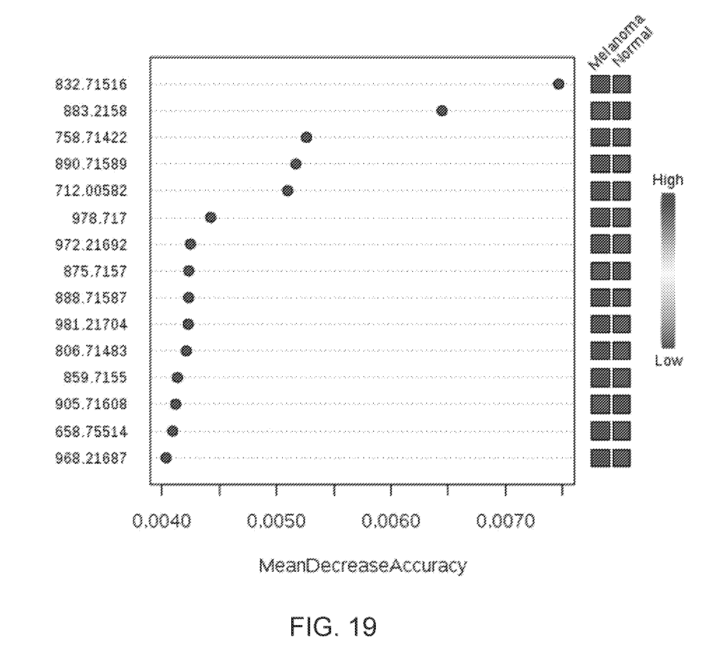

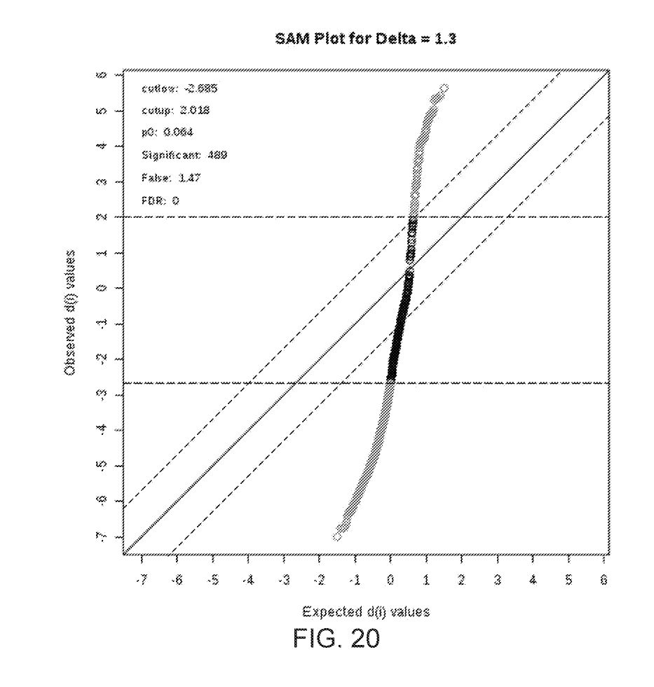

Direct analysis of the tissue was also performed utilizing a liquid micro-junction probe. This data showed principal component separation between the lipid portion of melanoma and normal skin tissue with principal component analysis (PCA) and partial least square-discriminant analysis (PLS-DA). Both the PCA scores plot (FIG. 16) and PLS-DA scores plot (FIG. 17) showed separation between melanoma and the control tissue samples within the first principal component. The top 30 features from the PLS-DA variable importance in projection (VIP) score plot (FIG. 18), top 15 features from the Random Forest mean decrease accuracy (MDA) plot (FIG. 19), and the SAM plot (FIG. 20) showing that 489 lipid features fall within the positive and negative standard deviation distribution estimated by random permutations of the dataset are provided here. The FDR rate reported is low for these 489 lipids indicating that they accurately define the differences between the melanoma and control tissue samples.

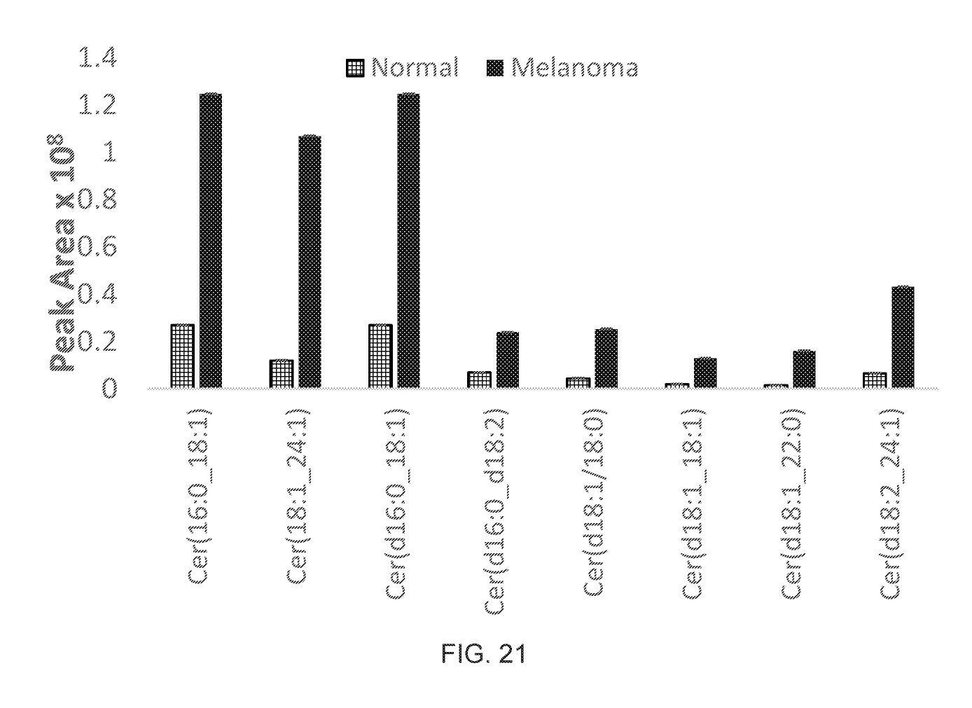

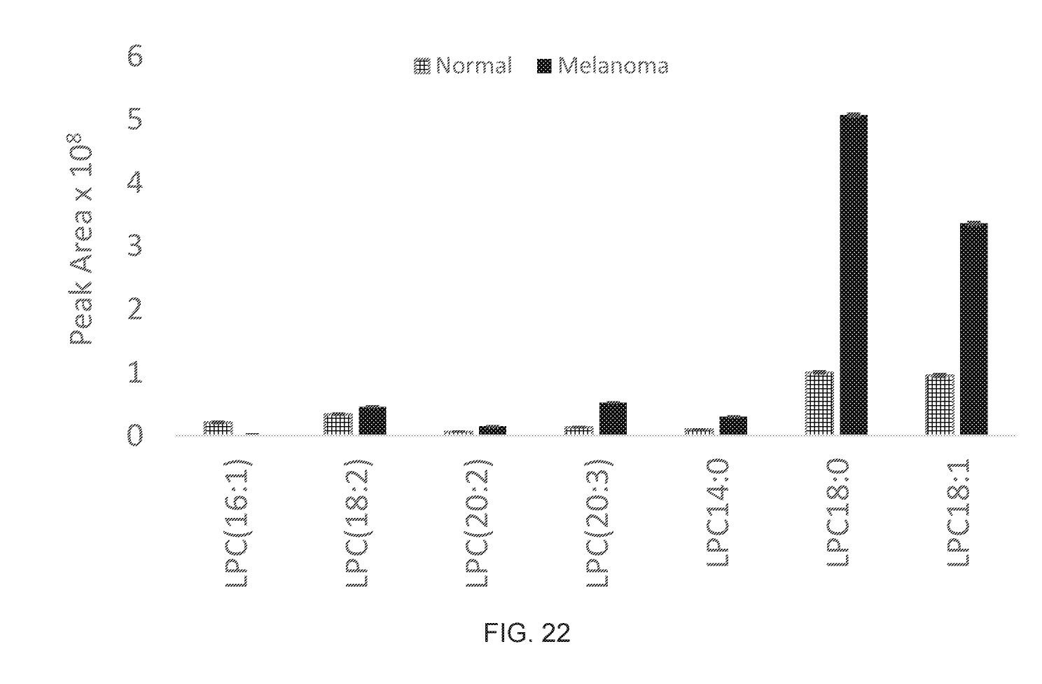

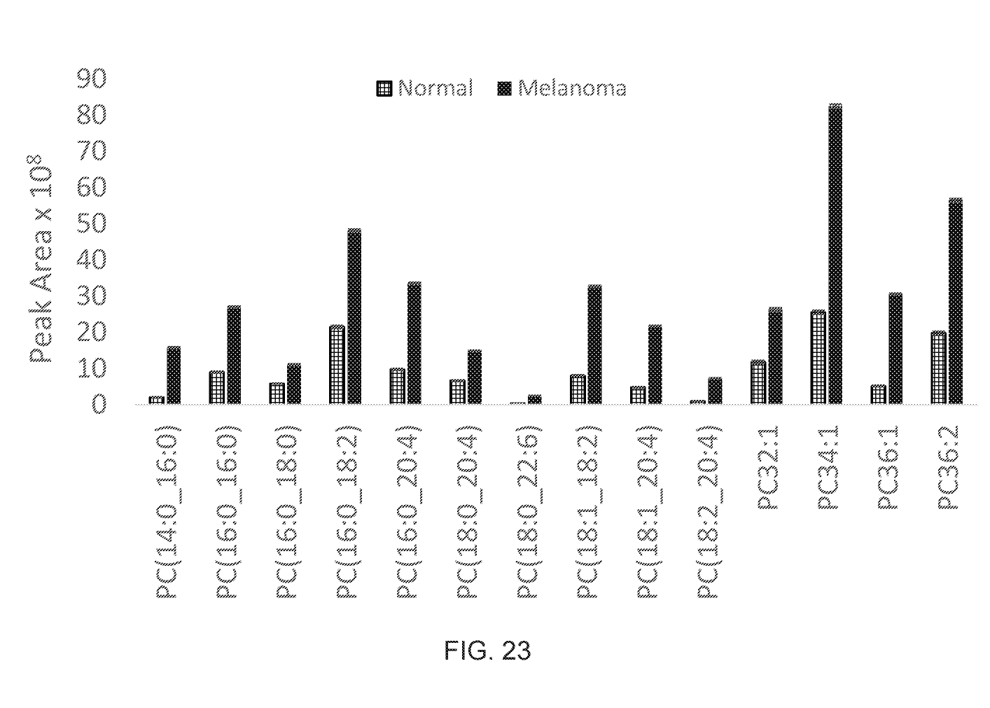

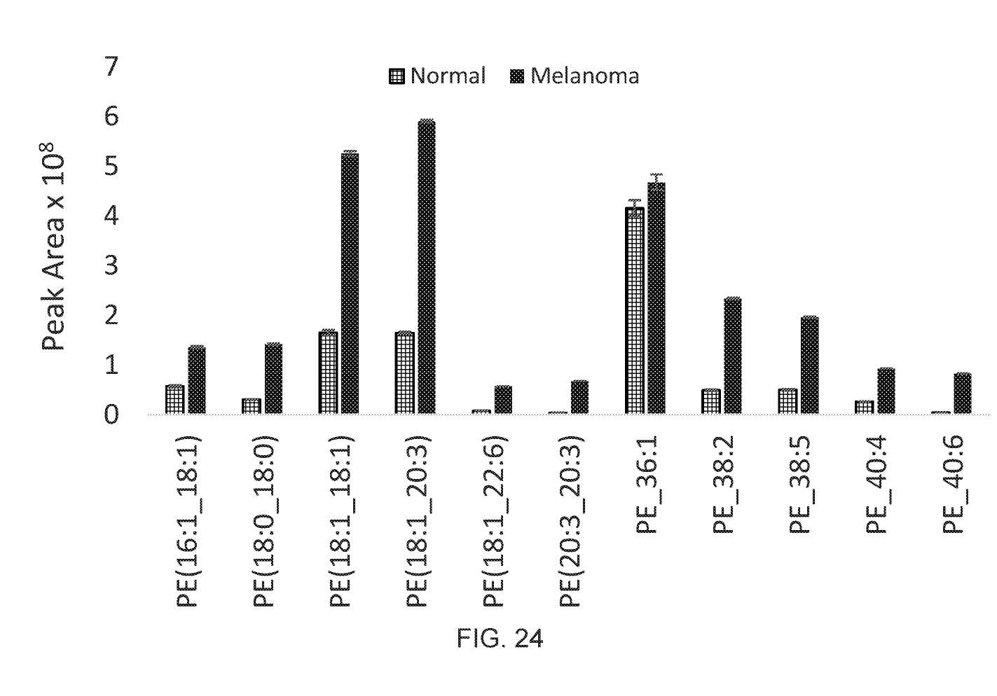

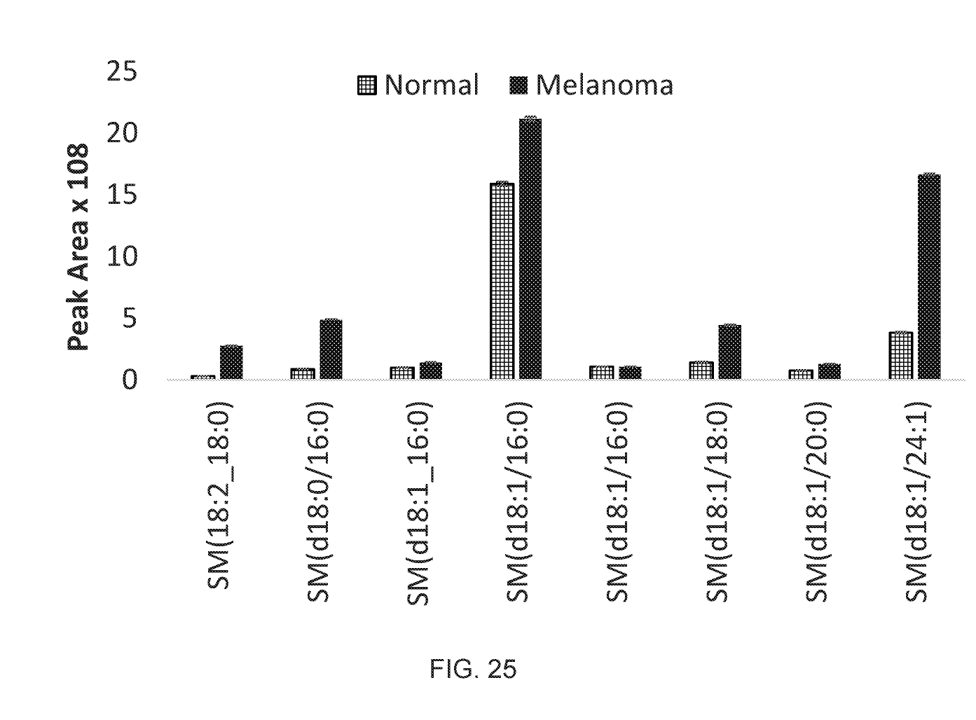

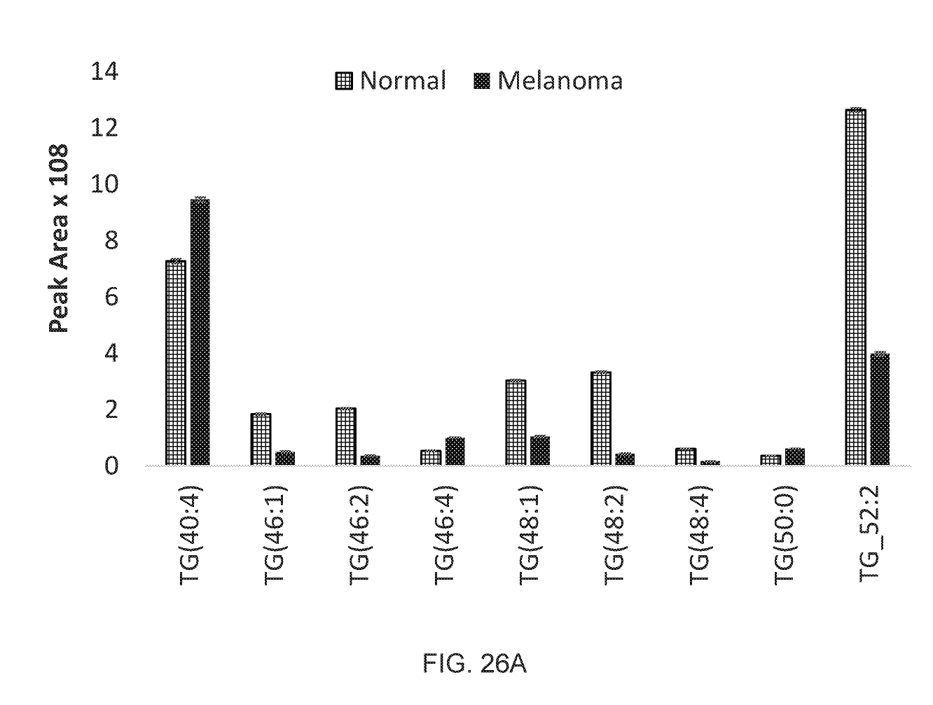

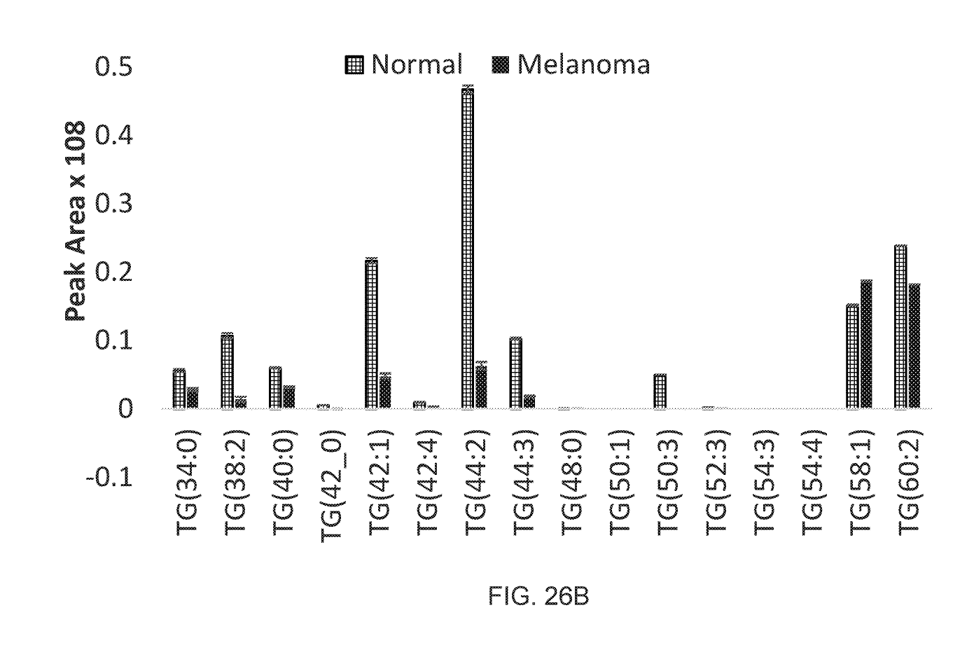

There was an observed increased in abundance of ceremides (Cer), phophatidylcholines (PC), lisophophatidylcholines (LPC), phosphatidylethanolamines (PE), and sphingomyelins (SM) in melanoma tissue vs normal tissue (FIGS. 21-25). The data from the lipid extracts also revealed notable trends in the relative intensities of distinct lipid classes. Conversely, there was an observed decrease in abundance of triacylglycerides (TAG) in melanoma vs normal tissues (FIGS. 26A-B).

Table 5, below is a compilation of some of the biomarkers identified in Examples 1-4, above, with the m/z values and compound names (where available). Number in parenthesis indicates in which example(s) the compound was identified.

TABLE 5

| | | Upregulated(+)/ |
|---|---|---|
| | | downregulated |
| | | (−) in melanoma |
| m/z value (Example) | Compound name | vs. normal |
| 71.013 (3) | Lactic acid | + |
| 86.096 (1) | | + |
| 90.055 (2) | | + |
| 107.049 (1) | Benzaldehyde | + |
| 109.065 (1) | | + |
| 116.070 (1) | Proline | + |
| 118.050 (3) | Succinic anhydride | − |
| 118.086 (2) | Betaine | + |
| 120.081 (1) | | + |
| 121.030 (3) | Benzoic acid | − |
| 132.102 (1) | Leucine (or Isoleucine) | + |
| 135.102 (1) | Polypropylene glycol | − |
| 138.066 (3) | Histidine | − |
| 145.122 (1) | Octanoic acid | + |
| 146.028 (3) | L-methionine S-oxide | + |
| 146.165 (1) | Spermidine | + |
| 150.059 (2) | Methionine | + |
| 158.118 (3) | Octadienoic acid | − |
| 164.056 (3) | 4-Hydroxy-L-glutamic acid | − |
| 166.086 (1 & 2) | Phenylalanine | + |
| 170.093/192.075 (3) | Methylhistidine | − |
| 171.078 (3) | Glycine-Proline | − |
| 175.071 (2) | Aminomuconic acid | − |
| 184.073/222.030/ | Phosphocholine | + |
| 228.063 (3) | | |
| 188.056 (3) | N-acetyl-L-glutamic acid | − |
| 188.071 (1) | | + |
| 188.176 (3) | Acetyl spermidine | + |
| 190.051 (3) | 5-Hydroxyindoleacetic acid | − |
| 191.089 (1) | | − |
| 192.069 (1) | | + |
| 194.042 (2) | Tetrahydrodipicolinate | − |
| 194.049 (3) | Thiopurine | + |
| 204.123 (1) | | + |
| 205.097 (1 & 2) | Tryptophan | + |
| 208.116 (1) | | − |
| 214.015 (3) | Carmustine | + |
| 219.080 (3) | N-formylmethionine | + |
| 225.097 (2) | | + |
| 231.098 (3) | 4-(glutamylamino) | + |
| | butanoate | |
| 239.065 (3) | Glutamylalanine | − |
| 240.121 (3) | Propionyl-L-carnitine | + |
| 245.043 (3) | Phosphatidylglycerol | + |
| 248.960 (3) | 5-(4-Chloro-3-hydroxy-1- | − |
| | butynyl)-2,2′-bithiophene | |
| 259.023 (3) | Ethyl glucuronide | − |
| 260.089 (3) | Aspartyl-gamma-glutamate | + |
| 262.104 (3) | Aspartyl-glutamine | + |
| 278.033 (3) | DOPA sulfate | + |
| 288.253 (1) | | − |
| 300.191 (3) | Alanine-Leucine-Proline | + |
| 309.226 (3) | Diacetyl spermine | + |
| 310.235 (1) | | − |
| 313.071 (3) | Skullcapflavone I | − |
| 329.234 (3) | Trihydroxyoctadecenoic | − |
| | acid | |
| 348.071 (2) | Adenosine monophosphate | − |
| 347.039/349.055 (3) | Inosine 5′-monophosphate | + |
| 361.164 (3) | Alanine-Glycine-Histidine- | − |
| | Proline | |
| 560.369 (4) | | − |
| 568.363 (4) | | + |
| 700.530 (4) | | + |
| 750.535 (4) | | + |
| 810.599 (4) | | − |
| 818.727 (4) | | − |
| 854.595 (4) | | + |
| 879.753 (4) | | − |
| 479.3926 (4) | Didodecyl thiobispropanoate | − |
| 484.4359 (4) | MG 24:0 | + |
| 594.4848 (4) | PC(O-12:0/O-12:0) | + |
| 677.5486 (4) | DG 42:6 | − |
| 781.5512 (4) | PE 38:4 | − |
| 798.6342 (4) | PC(O-14:0/22:0) | + |
| 804.5904 (4) | PE(P-20:0/22:6) | + |
| 817.6381 (4) | TG 50:9 | + |
| 819.5192 (4) | PA 42:7 | + |
| 820.5851 (4) | PC 39:6 | + |
| 821.6667 (4) | TG 50:7 | − |
| 822.5380 (4) | C18-OH Sulfatide | + |
| 832.7417 (4) | TG 49:3 | − |
| 845.5338 (4) | PA 44:8 | + |
| 878.5910 (4) | PC 40:6 | + |
| 879.7357 (4) | GlcCer(d18:1/26:1) | − |
| 890.6653 (4) | PC 44:6 | + |
| 898.7263 (4) | PC 44:2 | + |
| 917.8307 (4) | TG 59:4 | − |

### References

Kwak, J; Gallagher, M; Ozdener, M H; Wysocki, C; Goldsmith, B; lsamah, A; Faranda, A; Fakharzadeh, S S; Herlyn, M; Johnson, A T C; Preti, G. J Chromatogr B. 2013, 931, 90-96

Williams, H; Pembroke, A. Lancet 1989, 1, 734

Meij, T; Ben Larbi, I; Schee, M P; Lentferink, Y E; Paff, T; Terhaar sive Droste, J S; Mulder, C J; Bodegraven, A A; Boer, N. Int J Cancer. 2014, 134, 1132-1138

Buszewski, B; Rudnicka, J; Ligor, T; Walczak, M; Jezierski, T; Amann, A. Trends in Anal Chem. 2012, 38, 1-12

Amundsen, T; Sundstrom, S; Buvik, T; Gederaas, O A; Rune, H. Acta Oncol. 2014, 53, 307-315

“Can Bees be Trained to Sniff Out Cancer?” 2013: http://www.smithsonianmag.com

Abaffy, T; Moller, M G; Riemer, D D; Milikowski, C; DeFazio, R A. Metabolomics 2013, 9, 998-1008

Costanzo, M; Ulmer, C; Gravenstein, N; Yost R. Annual Pittcon Conference and Exposition, New Orleans, La. March 2015

Costanzo, M; Ulmer, C; Mohan, S; Gravenstein, N; Yost R. Proceedings of the 62nd ASMS Conference on Mass Spectrometry and Allied Topics, Baltimore, Md. June 2014

Wenting, L; Guangguo, T; Zhenyu, Z; Qiuli, C; Ziyang, L; Xin, D; Wei, Z; Wei, P; Yifeng, C. J Proteome Res. 2012, 11, 5109-5123

“Quant-iT Protein Assay Kit Manual” 2015: lifetechnologies.com

Wishart, D; Jewison, T; et al. Nucleic Acids Res. 2013, 41, D801-807

Xia, J; Mandal, R; Sinelnikov, I; Broadhurst, D; Wishart, D S. Nuc Acids Rec. 2012, 40, W127-W133

Smith, C; Want E; et al. Anal Chem. 2006, 78, 779-787

m/zCloud: mzcloud.org

METLIN: metlin.scripps.edu

Ismail, M. A.; Arafa, R. K.; Youssef, M. M.; EI-Sayed, W. Drug Design, Development and Therapy. 2014, 8, 1659-1672.

Hu, K.; Du, K.; Tang, G.; Yao, S.; Wang, H.; Liang, X.; Yao, B.; Huang, T.; Zang, L. PLoS ONE. 2014, 9, e93262.

Randhawa, M.; Sangar, V.; Tucker-Samaras, S.; Southall, M. PLoS ONE. 2014, 9, e90367.

Darvas, Z.; Hegyesi, H.; László, V.; Bencsáth, M.; Falus, A.; Haak-Frendscho, M.; Kárpáti, S.; Hoffman, R. L.; Szalai, C.; Fürész, J.; Timár, J.; Bata-Csörgö, Z.; Szabad, G.; Pivarcsi, A.; Pállinger, É; Kemény, L.; Horváth, A.; Dobozy, A. J.Invest.Dermatol. 2000, 115, 345-352.

Young, S. E.; Griffin, A. C.; Milner, A. N.; Stehlin, J. S., Jr. Cancer Res. 1967, 27, 15-17.

Bianchi-Smiraglia, A.; Wawrzyniak, J. A.; Bagati, A.; Marvin, E. K.; Ackroyd, J.; Moparthy, S.; Bshara, W.; Fink, E. E.; Foley, C. E.; Morozevich, G. E.; Berman, A. E.; Shewach, D. S.; Nikiforov, M. A. Cell Death Differ. 2015, 22, 1858-1864.

Long, M. D.; Martin, C. F.; Pipkin, C. A.; Herfarth, H. H.; Sandler, R. S.; Kappelman, M. D. Gastroenterology. 2012, 143, 390-399.e1.

Abbas, A. M.; Almukhtar, R. M.; Loftus, E. V.; Lichtenstein, G. R.; Khan, N. Am. J. Gastroenterol. 2014, 109, 1781-1793.

Morvan, D.; Demidem, A.; Madelmont, J. C. Pathol.Biol.(Paris). 2003, 51, 256-259.

Takahashi, Y.; Horio, H.; Sakaguchi, K.; Hiramatsu, K.; Kawakita, M. BMC Cancer. 2015, 15, 65.

Seiler, N.; Graham, A.; Bartholeyns, J. Cancer Res. 1981, 41, 1572-1573.

1-26. (canceled)

27. A method of detecting melanoma in a host, the method comprising:
non-invasively obtaining a sample from a skin lesion of a host, wherein the lesion is suspected of including melanoma;
analyzing the sample with a chemical analysis technique and determining the presence, amount, or both in the sample of a set of identified melanoma biomarkers selected based on multivariate data analysis of melanoma and non-melanoma samples;
providing a biomarker signature of the host sample based on the chemical analysis of the sample, wherein the biomarker signature indicates the presence, amount, or both of one or more of the melanoma biomarkers;
comparing the biomarker signature of the host sample to a biomarker signature of a control sample; and
making a preliminary patient diagnosis of melanoma based on the comparison of the host sample biomarker signature and the control sample biomarker signature.

28. The method of claim 27, wherein the chemical analysis technique is selected from the group consisting of: mass spectrometry, liquid chromatography, gas chromatography, ion mobility spectrometry, and high-field asymmetric waveform ion mobility spectrometry.

29. The method of claim 27, wherein the melanoma biomarkers comprise volatile compounds, lipid compounds, or both.

30. The method of claim 27, wherein the melanoma biomarkers are selected from the group of compounds consisting of: lactic acid, a compound correlated to an m/z value 86.096, a compound correlated to an m/z value 90.055, benzaldehyde, a compound correlated to an m/z value 109.065, proline, succinic anhydride, betaine, a compound correlated to an m/z value 120.081, benzoic acid, leucine or isoleucine, propylene glycol, histidine, octanoic acid, L-methionine S-oxide, spermidine, methionine, octadienoic acid, 4-hydroxy-L-glutamic acid, phenylalanine, methylhistidine, glycine-proline, aminomuconic acid, phosphocholine, N-acetyl-L-glutamic acid, a compound correlated to an m/z value 188.071, acetyl spermidine, 5-hydroxyindoleacetic acid, a compound correlated to the m/z value 191.089, a compound correlated to an m/z value 192.069, tetrahydrodipicolinate, thiopurine, a compound correlated to an m/z value 204.123, tryptophan, a compound correlated to an m/z value 208.116, carmustine, n-formylmethionine, a compound correlated to an m/z value 225.097, 4-(glutamylamino) butanoate, glutamylalanine, propionyl-L-carnitine, phosphatidylglycerol, 5-(4-Chloro-3-hydroxy-1-butynyl)-2,2′-bithiophene, ethyl glucuronide, aspartyl-gamma-glutamate, aspartyl-glutamine, DOPA sulfate, a compound correlated to an m/z value 288.253, alanine-leucine-proline, diacetyl spermine, a compound correlated to an m/z value 310.235, skullcapflavone I, trihydroxyoctadecenoic acid, adenosine monophosphate, inosine 5′-monophosphate, alanine-glycine-histidine-proline, and the group of lipids corresponding to the group of m/z values consisting of: 560.369, 568.363, 700.530, 750.535, 810.599, 818.727, 854.595, and 879.735.

31. The method of claim 27, wherein obtaining the host sample comprises provoking an emission from the skin lesion of the host, and wherein provoking the emission comprises a technique selected from the group consisting of: physical contact with the skin lesion, suction/aspiration of the skin lesion or the air surrounding the skin lesion, and particle bombardment of the skin lesion.

32. The method of claim 31, wherein the emission comprises a volatile emission.

33. The method of claim 27, wherein the sample obtained from the host is not subject to further processing steps prior to analysis.

34. The method of claim 27, further comprising validating the determined diagnosis by performing one or both of the following:
analyzing the host sample with one or more additional chemical analysis procedures selected from: liquid chromatography and high-resolution mass spectrometry, and
obtaining and analyzing a second sample from the skin lesion of the host with one or more additional chemical analysis procedures selected from: liquid chromatography and high-resolution mass spectrometry.

35. The method of claim 27, wherein the analysis of the sample is performed with a portable ion-mobility separation (IMS) mass spectrometer or a high-field asymmetric ion mobility spectrometer (FAIMS).

36. The method of claim 27, further comprising:
a) determining whether at least one compound selected from the group of compounds from group (A) is overexpressed in the host volatile sample, wherein group (A) comprises compounds selected from the group consisting of: lactic acid, a compound correlated to an m/z value 86.096, 90.055, benzaldehyde, a compound correlated to an m/z value 109.065, proline, betaine, the compound correlated to an m/z value 120.081, leucine or isoleucine, octanoic acid, L-methionine S-oxide, spermidine, methionine, phenylalanine, phosphocholine, a compound correlated to an m/z value 188.071, acetyl spermidine, a compound correlated to an m/z value 192.069, thiopurine, a compound correlated to an m/z value 204.123, tryptophan, carmustine, n-formylmethionine, a compound correlated to an m/z value 225.097, 4-(glutamylamino) butanoate, propionyl-L-carnitine, phosphatidylglycerol, aspartyl-gamma-glutamate, aspartyl-glutamine, DOPA sulfate, alanine-leucine-proline, diacetyl spermine, inosine 5′-monophosphate, a lipid correlated to an m/z value 568.363, a lipid correlated to an m/z value 700.530, a lipid correlated to an m/z value 750.535, and a lipid correlated to an m/z value 854.595, or
b) determining whether at least one compound selected from the group of compounds from group (B) is underexpressed in the host volatile sample, wherein group (B) comprises compounds from the group consisting of: having m/z values consisting of: succinic anhydride, benzoic acid, propylene glycol, histidine, octadienoic acid, 4-hydroxy-L-glutamic acid, methylhistidine, glycine-proline, aminomuconic acid, N-acetyl-L-glutamic acid, 5-hydroxyindoleacetic acid, a compound correlated to an m/z value 191.089, tetrahydrodipicolinate, a compound correlated to an m/z value 208.116, glutamylalanine, 5-(4-chloro-3-hydroxy-1-butynyl)-2,2′-bithiophene, ethyl glucuronide, 288.253, a compound correlated to an m/z value 310.235, skullcapflavone I, trihydroxyoctadecenoic acid, adenosine monophosphate, alanine-glycine-histidine-proline, a lipid correlated to an m/z value 560.369, a lipid correlated to an m/z value 810.599, a lipid correlated to an m/z value 818.727, and a lipid correlated to an m/z value 879.735;
or a) and b), and
diagnosing the host as having melanoma or a risk of developing melanoma if at least one of the compounds from group (A) is overexpressed, at least one of the compounds from group (B) is underexpressed, or both, when compared to a mean expression level of the compound(s) in one or more control samples.

37. The method of any of claims 27, wherein the biomarker signature indicates the presence, amount, or both of two or more of the melanoma biomarkers.

38. A system for real-time detection of melanoma in a host, the system comprising:
a device for provoking an emission from a skin lesion of a host to produce an emission sample from the host, wherein the lesion is suspected of including melanoma;
a sample reservoir for containing or capturing the emission sample, wherein the sample reservoir is configured to interface with a mass spectrometry device;
a mass spectrometry device capable of analyzing the chemical content of the emission sample; and
a signal processing mechanism in operative communication with the mass spectrometry device, the signal processing mechanism having data transfer and evaluation software protocols configured to transform raw data from the mass spectrometry device into information regarding melanoma biomarkers, wherein the information comprises one or more of the presence, absence, and amounts of the melanoma biomarkers in the emission sample.

39. The system of claim 38, wherein the signal processing mechanism produces a biomarker signature corresponding to the host sample.

40. The system of claim 38, wherein the signal processing mechanism compares the biomarker signature corresponding to the host sample to a biomarker signature of a control sample.

41. The system of claim 38, wherein the signal processing mechanism outputs a preliminary diagnosis of melanoma based on the comparison of the host sample biomarker signature and the control sample biomarker signature.

42. A method of diagnosing or monitoring melanoma in a host, the method comprising:
obtaining a sample from a skin lesion of the host, wherein the lesion is suspected of including melanoma;
analyzing the sample with a chemical analysis technique selected from the group consisting of: mass spectrometry, liquid chromatography, gas chromatography, ion mobility spectrometry, and high-field asymmetric waveform ion mobility spectrometry to determine a molecular signature of the sample, a lipid signature of the sample, or both;
detecting the presence, amount, or both of one or more molecular melanoma biomarkers, lipid melanoma biomarkers, or both, from the molecular signature, lipid signature, or both, of the sample,
wherein the molecular melanoma biomarkers are selected from the group of compounds having m/z values selected from the group consisting of: lactic acid, a compound correlated to an m/z value 86.096, a compound correlated to an m/z value 90.055, benzaldehyde, a compound correlated to an m/z value 109.065, proline, succinic anhydride, betaine, a compound correlated to an m/z value 120.081, benzoic acid, leucine or isoleucine, propylene glycol, histidine, octanoic acid, L-methionine S-oxide, spermidine, methionine, octadienoic acid, 4-hydroxy-L-glutamic acid, phenylalanine, methylhistidine, glycine-proline, aminomuconic acid, phosphocholine, N-acetyl-L-glutamic acid, a compound correlated to an m/z value 188.071, acetyl spermidine, 5-hydroxyindoleacetic acid, a compound correlated to the m/z value 191.089, a compound correlated to an m/z value 192.069, tetrahydrodipicolinate, thiopurine, a compound correlated to an m/z value 204.123, tryptophan, a compound correlated to an m/z value 208.116, carmustine, n-formylmethionine, a compound correlated to an m/z value 225.097, 4-(glutamylamino) butanoate, glutamylalanine, propionyl-L-carnitine, phosphatidylglycerol, 5-(4-Chloro-3-hydroxy-1-butynyl)-2,2′-bithiophene, ethyl glucuronide, aspartyl-gamma-glutamate, aspartyl-glutamine, DOPA sulfate, a compound correlated to an m/z value 288.253, alanine-leucine-proline, diacetyl spermine, a compound correlated to an m/z value 310.235, skullcapflavone I, trihydroxyoctadecenoic acid, adenosine monophosphate, inosine 5′-monophosphate, alanine-glycine-histidine-proline, and the group of lipids corresponding to the group of m/z values consisting of: 560.369, 568.363, 700.530, 750.535, 810.599, 818.727, 854.595, or 879.735; and
wherein the lipid melanoma biomarkers are selected from the group of lipids corresponding to the group of m/z values consisting of: 560.369, 568.363, 700.530, 750.535, 810.599, 818.727, 854.595, 879.735, 484.436, 594.485, 798.634, 804.590, 817.638, 819.519, 820.585, 822.538, 845.534, 878.591, 890.665, 898.726, 479.393, 677.549, 781.551, 821.667, 832.742, 879.736, and 917.831
comparing the amount of the one or more molecular melanoma biomarkers, lipid melanoma biomarkers, or both, from the host sample an amount of the same one or more molecular melanoma biomarkers, lipid melanoma biomarkers, or both, from a molecular signature of a control sample, and
making a preliminary diagnosis of melanoma when one or more molecular melanoma biomarkers, lipid melanoma biomarkers, or both, are present in a concentration significantly greater than or significantly less than the concentration of the same compound in the control sample.

43. The method of claim 42, wherein comparing the amount of biomarkers comprises comparing the amount of two or more molecular melanoma biomarkers from the host sample to an amount of the same two or more molecular melanoma biomarkers from a molecular signature of a control sample, and wherein a preliminary diagnosis of melanoma is made when two or more molecular melanoma biomarkers are present in a concentration significantly greater than or significantly less than the concentration in the control sample.

44. The method of claim 42, further comprising determining a potential diagnosis of melanoma when one or more lipid melanoma biomarkers selected from the group of lipid melanoma biomarkers correlated with the following m/z values are present in a greater amount in the host sample as compared to the amount the control sample: 568.363, 700.530, 750.535, 854.595, 484.436, 594.485, 798.634, 804.590, 817.638, 819.519, 820.585, 822.538, 845.534, 878.591, 890.665, and 898.726.

45. The method of claim 42, further comprising determining a potential diagnosis of melanoma when one or more lipid melanoma biomarkers selected from the following are present in a lower amount in the host sample as compared to the amount in the control sample: 560.369, 810.599, 818.727, 879.735 479.3926, 677.5486, 781.5512, 821.6667, 832.7417, 879.7357, 917.8307.
